(19) United States
(12) Patent Application Publication
(10) Pub. No.: US 2021/0309669 A1
(43) Pub. Date: Oct. 7, 2021

(54) Heterocyclic Spiro Compounds As MAGL Inhibitors

(71) Applicant: Pfizer Inc., New York, NY (US)

(72) Inventors: Michael Aaron Brodney, Newton, MA (US); Christopher Ryan Butler, Canton, MA (US); Laura Ann McAllister, Arlington, MA (US); Steven Victor O'Neil, East Lyme, CT (US)

(73) Assignee: Pfizer Inc., New York, NY (US)

(21) Appl. No.: 17/260,247

(22) PCT Filed: Jul. 10, 2019
(86) PCT No.: PCT/IB2019/055893

§ 371 Date: Jan. 14, 2021

Related U.S. Application Data
(60) Provisional application No. 62/700,386, filed on Jul. 19, 2018.

(51) Int. Cl. C07D 491/107

(52) U.S. Cl. CPC C07D 491/107

(57) ABSTRACT

The present invention provides, in part, a compound selected from the group consisting of:
1,1,1,3,3,3-hexafluoropropan-2-yl (3R)-3-{[(cyclopropylmethyl)sulfonyl]amino}-1-oxa-8-azaspiro[4.5]decane-8-carboxylate;
1,1,1,3,3,3-hexafluoropropan-2-yl (3R)-3-[(cyclopropylsulfonyl)amino]-1-oxa-8-azaspiro[4.5]decane-8-carboxylate;
1,1,1,3,3,3-hexafluoropropan-2-yl 3-{[(cyclopropylmethyl)sulfonyl]amino}-1-oxa-9-azaspiro[5.5]undecane-9-carboxylate;
1,1,1,3,3,3-hexafluoropropan-2-yl 3-{[(cyclopropylmethyl)sulfonyl]amino}-1-oxa-9-azaspiro[5.5]undecane-9-carboxylate, ENT-1; and
1,1,1,3,3,3-hexafluoropropan-2-yl 3-{[(cyclopropylmethyl)sulfonyl]amino}-1-oxa-9-azaspiro[5.5]undecane-9-carboxylate, ENT-2,
and pharmaceutically acceptable salts thereof; processes for the preparation of; intermediates used in the preparation of; and compositions containing such compounds or salts, and their uses for treating MAGL-mediated diseases and disorders including, e.g., pain, an inflammatory disorder, depression, anxiety, Alzheimer's disease, a metabolic disorder, steatohepatitis [e.g. nonalcoholic Steatohepatitis (NASH)], stroke, or cancer.

### FIELD OF THE INVENTION

The present invention relates to novel heterocyclic spiro compounds, which are monoacylglycerol lipase (MAGL) inhibitors, pharmaceutical compositions thereof, and uses thereof in the treatment of MAGL-mediated disorders such as pain, an inflammatory disorder, depression, anxiety, Alzheimer's disease, a metabolic disorder, stroke, or cancer.

### BACKGROUND OF THE INVENTION

MAGL is the principal enzyme responsible for the in vivo degradation of 2-arachidonoyl glycerol (2-AG), an endogenous ligand of the cannabinoid receptors (e.g., CB1 and CB2). See e.g., Patel, J. Z. et al., “Loratadine analogues as MAGL inhibitors,” Bioorg. Med. Chem. Lett., 2015, 25(7):1436-42; Mechoulam, R. et al., “Identification of an endogenous 2-monoglyceride, present in canine gut, that binds to cannabinoid receptors” Biochem. Pharmacol., 50 (1995), 83-90; Sugiura, T. et al., “2-Arachidonoylglycerol: a possible endogenous cannabinoid receptor ligand in brain,” Biochem. Biophys. Res. Commun., 215 (1995), 89-97.

MAGL inhibitors are potentially useful for the treatment of a MAGL-mediated disease or disorder. Examples of MAGL-mediated diseases or disorders include a metabolic disorder (e.g., obesity); vomiting or emesis; nausea; an eating disorder (e.g., anorexia or bulimia); neuropathy (e.g., diabetic neuropathy, pellagric neuropathy, alcoholic neuropathy, Beriberi neuropathy); burning feet syndrome; a neurodegenerative disorder [multiple sclerosis (MS), Parkinson's disease (PD), Huntington's disease, Alzheimer's disease, amyotrophic lateral sclerosis (ALS), epilepsy, a sleep disorder, Creutzfeldt-Jakob disease (CJD), or prion disease]; a cardiovascular disease (e.g., hypertension, dyslipidemia, atherosclerosis, cardiac arrhythmias, or cardiac ischemia); osteoporosis; osteoarthritis; schizophrenia; depression; bipolar disease; tremor; dyskinesia; dystonia; spasticity; Tourette's syndrome; sleep apnea; hearing loss; an eye disease (e.g., glaucoma, ocular hypertension, macular degeneration, or a disease arising from elevated intraocular pressure); cachexia; insomnia; meningitis; sleeping sickness; progressive multifocal leukoencephalopathy; De Vivo disease; cerebral edema; cerebral palsy; withdrawal syndrome [alcohol withdrawal syndrome, antidepressant discontinuation syndrome, antipsychotic withdrawal syndrome, benzodiazepine withdrawal syndrome, cannabis withdrawal, neonatal withdrawal, nicotine withdrawal, or opioid withdrawal]; traumatic brain injury; spinal cord injury; seizures; excitotoxin exposure; ischemia [stroke, hepatic ischemia or reperfusion, CNS ischemia or reperfusion]; liver fibrosis, iron overload, cirrhosis of the liver; a lung disorder [asthma, allergies, COPD, chronic bronchitis, emphysema, cystic fibrosis, pneumonia, tuberculosis, pulmonary edema, lung cancers, acute respiratory distress syndrome, intersitital lung disease (ILD), sarcoidosis, idiopathic pulmonary fibrosis, pulmonary embolism, pleural effusion, or mesothelioma]; a liver disorder [acute liver failure, Alagille syndrome, hepatitis, enlarged liver, Gilbert's syndrome, liver cysts, liver hemangioma, fatty liver disease, steatohepatitis [e.g. nonalcoholic Steatohepatitis (NASH)], primary sclerosing cholangitis, fascioliasis, primary bilary cirrhosis, Budd-Chiari syndrome, hemochromatosis, Wilson's disease, or transthyretin-related hereditary amyloidosis], stroke [e.g., ischemic stroke; hemorrhagic stroke]; subarachnoid hemorrhage; vasospasm; AIDS wasting syndrome; renal ischemia; a disorder associated with abnormal cell growth or proliferation [e.g., a benign tumor or cancer such as benign skin tumor, brain tumor, papilloma, prostate tumor, cerebral tumor (glioblastoma, medulloepithelioma, medulloblastoma, neuroblastoma, astrocytoma, astroblastoma, ependymoma, oligodendroglioma, plexus tumor, neuroepithelioma, epiphyseal tumor, ependymoblastoma, malignant meningioma, sarcomatosis, melanoma, schwannoma), melanoma, metastatic tumor, kidney cancer, bladder cancer, brain cancer, glioblastoma (GBM), gastrointestinal cancer, leukemia or blood cancer]; an autoimmune disease [e.g., psoriasis, lupus erythematosus, Sjögren's syndrome, ankylosing spondylitis, undifferentiated spondylitis, Behcet's disease, hemolytic anemia, graft rejection]; an inflammatory disorder [e.g., appendicitis, bursitis, colitis, cystitis, dermatitis, phlebitis, rhinitis, tendonitis, tonsillitis, vasculitis, acne vulgaris, chronic prostatitis, glomerulonephritis, hypersensitivities, IBS, pelvic inflammatory disease, sarcoidosis, HIV encephalitis, rabies, brain abscess, neuroinflammation, inflammation in the central nervous system (CNS)]; a disorder of the immune system (e.g., transplant rejection or celiac disease); post-traumatic stress disorder (PTSD); acute stress disorder; panic disorder; substance-induced anxiety; obsessive-compulsive disorder (OCD); agoraphobia; specific phobia; social phobia; anxiety disorder; attention deficit disorder (ADD); attention deficit hyperactivity disorder (ADHD); Asperger's syndrome; pain [e.g., acute pain; chronic pain; inflammatory pain; visceral pain; post-operative pain; migraine; lower back pain; joint pain; abdominal pain; chest pain; postmastectomy pain syndrome; menstrual pain; endometriosis pain; pain due to physical trauma; headache; sinus headache; tension headache arachnoiditis, herpes virus pain, diabetic pain; pain due to a disorder selected from: osteoarthritis, rheumatoid arthritis, osteoarthritis, spondylitis, gout, labor, musculoskeletal disease, skin disease, toothache, pyresis, burn, sunburn, snake bite, venomous snake bite, spider bite, insect sting, neurogenic bladder, interstitial cystitis, urinary tract infection (UTI), rhinitis, contact dermatitis/hypersensitivity, itch, eczema, pharyngitis, mucositis, enteritis, irritable bowel syndrome (IBS), cholecystitis, and pancreatitis; neuropathic pain (e.g., neuropathic low back pain, complex regional pain syndrome, post trigeminal neuralgia, causalgia, toxic neuropathy, reflex sympathetic dystrophy, diabetic neuropathy, chronic neuropathy from chemotherapeutic agent, or sciatica pain)]; a demyelinating disease [e.g., multiple sclerosis (MS), Devic's disease, CNS neuropathies, central pontine myelinolysis, syphilitic myelopathy, leukoencephalopathies, leukodystrophies, Guillain-Barre syndrome, chronic inflammatory demyelinating polyneuropathy, anti-myelin-associated glycoprotein (MAG) peripheral neuropathy, Charcot-Marie-Tooth disease, peripheral neuropathy, myelopathy, optic neuropathy, progressive inflammatory neuropathy, optic neuritis, transverse myelitis]; and cognitive impairment [e.g., cognitive impairment associated with Down's syndrome; cognitive impairment associated with Alzheimer's disease; cognitive impairment associated with PD; mild cognitive impairment (MCI), dementia, post-chemotherapy cognitive impairment (PCCI), postoperative cognitive dysfunction (POCD)]. See e.g., U.S. Pat. Nos. 8,415,341, 8,835,418, or U.S. Pat. No. 8,772,318.

There continues to be a need for alternative MAGL inhibitors.

### SUMMARY OF THE INVENTION

The present invention provides, in part, a novel compound selected from the group consisting of:
1,1,1,3,3,3-hexafluoropropan-2-yl (3R)-3-{[(cyclopropylmethyl)sulfonyl]amino}-1-oxa-8-azaspiro[4.5]decane-8-carboxylate;
1,1,1,3,3,3-hexafluoropropan-2-yl (3R)-3-[(cyclopropylsulfonyl)amino]-1-oxa-8-azaspiro[4.5]decane-8-carboxylate;
1,1,1,3,3,3-hexafluoropropan-2-yl 3-{[(cyclopropylmethyl)sulfonyl]amino}-1-oxa-9-azaspiro[5.5]undecane-9-carboxylate;
1,1,1,3,3,3-hexafluoropropan-2-yl 3-{[(cyclopropylmethyl)sulfonyl]amino}-1-oxa-9-azaspiro[5.5]undecane-9-carboxylate, ENT-1; and
1,1,1,3,3,3-hexafluoropropan-2-yl 3-{[(cyclopropylmethyl)sulfonyl]amino}-1-oxa-9-azaspiro[5.5]undecane-9-carboxylate, ENT-2,

or a pharmaceutically acceptable salt thereof.

In some embodiments, the present invention provides a compound that is 1,1,1,3,3,3-hexafluoropropan-2-yl (3R)-3-{[(cyclopropylmethyl)sulfonyl]amino}-1-oxa-8-azaspiro[4.5]decane-8-carboxylate, or a pharmaceutically acceptable salt thereof. In some embodiments, the present invention provides the compound of 1,1,1,3,3,3-hexafluoropropan-2-yl (3R)-3-{[(cyclopropylmethyl)sulfonyl]amino}-1-oxa-8-azaspiro[4.5]decane-8-carboxylate. In some embodiments, the present invention provides a pharmaceutically acceptable salt of 1,1,1,3,3,3-hexafluoropropan-2-yl (3R)-3-{[(cyclopropylmethyl)sulfonyl]amino}-1-oxa-8-azaspiro[4.5]decane-8-carboxylate.

In some embodiments, the present invention provides a crystal form of anhydrous (anhydrate) 1,1,1,3,3,3-hexafluoropropan-2-yl (3R)-3-{[(cyclopropylmethyl)sulfonyl]amino}-1-oxa-8-azaspiro[4.5]decane-8-carboxylate. In some further embodiments, the crystal form of anhydrous (an hydrate) 1,1,1,3,3,3-hexafluoropropan-2-yl (3R)-3-{[(cyclopropylmethyl)sulfonyl]amino}-1-oxa-8-azaspiro[4.5]decane-8-carboxylate is Form I, that is characterized according to its unique solid state signatures with respect to, for example, powder X-ray diffraction (PXRD), differential scanning calorimetry (DSC), and/or other solid state methods described herein. Further characterization with respect to water or solvent content of the crystal forms can be gauged by any of various routine methods such as thermogravimetric analysis (TGA), dynamic vapor sorption (DVS), DSC and other techniques described herein.

In some embodiments, the present invention provides a crystal form of anhydrous (anhydrate) 1,1,1,3,3,3-hexafluoropropan-2-yl (3R)-3-{[(cyclopropylmethyl)sulfonyl]amino}-1-oxa-8-azaspiro[4.5]decane-8-carboxylate, designated herein as “Form I,” having a powder X-ray diffraction pattern substantially as depicted in FIG. 4. In some embodiments, the anhydrous crystal form of the invention (Form I) is substantially free of water or other organic solvent. A list of diffraction peaks expressed in terms of the degree 2θ and relative intensities with a relative intensity of ≥4.0% is provided above in Table 1.

TABLE 1

| Peak position [2 theta; | |
|---|---|
| degree (°)] | Intensity (%) |
| 5.2 | 20.6 |
| 10.2 | 100 |
| 13.5 | 12.9 |
| 17.7 | 38.2 |
| 18.0 | 13.3 |
| 18.4 | 17.2 |
| 18.8 | 12.5 |
| 19.9 | 9.5 |
| 20.4 | 20.3 |
| 21.8 | 6.8 |
| 22.3 | 5.7 |
| 23.4 | 8.0 |
| 24.7 | 7.7 |
| 27.1 | 5.8 |
| 29.1 | 6.1 |
| 29.6 | 6.3 |
| 35.7 | 4.0 |

In some embodiments, Form I exhibits a powder X-ray diffraction pattern comprising at least two characteristic peaks, in terms of 2θ, selected from at 5.2±0.2°; 10.2±0.2°; 13.5±0.2°; 17.7±0.2°; 18.4±0.2°; and 20.4±0.2°. In some embodiments, Form I exhibits a powder X-ray diffraction pattern comprising at least three characteristic peaks, in terms of 2θ, selected from at 5.2±0.2°; 10.2±0.2°; 13.5±0.2°; 17.7±0.2°; 18.4±0.2°; and 20.4±0.2°. In some embodiments, Form I exhibits a powder X-ray diffraction pattern comprising at least four characteristic peaks, in terms of 2θ, selected from at 5.2±0.2°; 10.2±0.2°; 13.5±0.2°; 17.7±0.2°; 18.4±0.2°; and 20.4±0.2°. In some embodiments, Form I exhibits a powder X-ray diffraction pattern comprising at least five characteristic peaks, in terms of 2θ, selected from at 5.2±0.2°; 10.2±0.2°; 13.5±0.2°; 17.7±0.2°; 18.4±0.2°; and 20.4±0.2°.

In some embodiments, Form I exhibits a powder X-ray diffraction pattern comprising characteristic peaks, in terms of 2θ, at 5.2±0.2° and 10.2±0.2°.

In some embodiments, Form I exhibits a powder X-ray diffraction pattern comprising peaks, in terms of 2θ, at 5.2±0.2°; 10.2±0.2°; and 13.5±0.2°. In some further embodiments, Form I exhibits the X-ray powder diffraction pattern further comprises at least one peak, in terms of 2θ, selected from at 17.7±0.2°; 18.4±0.2°; and 20.4±0.2°.

In some embodiments, Form I exhibits a powder X-ray diffraction pattern comprising peaks, in terms of 2θ, at 5.2±0.2°; 10.2±0.2°; 13.5±0.2°; and 17.7±0.2°.

In some embodiments, Form I exhibits a powder X-ray diffraction pattern comprising peaks, in terms of 2θ, at 5.2±0.2°; 10.2±0.2°; 13.5±0.2°; 17.7±0.2°; and 20.4±0.2°.

In some embodiments, Form I exhibits a powder X-ray diffraction pattern comprising peaks, in terms of 2θ, at 5.2±0.2°; 10.2±0.2°; 13.5±0.2°; 17.7±0.2°; 18.4±0.2°; and 20.4±0.2°. In some further embodiments, Form I exhibits the X-ray powder diffraction pattern further comprises at least one peak, in terms of 2θ, selected from at 18.0±0.2°; 18.8±0.2°; 19.9±0.2°; and 21.8±0.2°.

In some embodiments, Form I exhibits a powder X-ray diffraction pattern substantially as shown in FIG. 4.

As is well known in the art of powder diffraction, the relative intensities of the peaks (reflections) can vary, depending upon the sample preparation technique, the sample mounting procedure and the particular instrument employed. Moreover, instrument variation and other factors can affect the 2-theta values. Therefore, the XRPD peak assignments can vary by plus or minus about 0.2°.

Data of Table 2 pertaining to water content of the crystal form of Form I, shows that the anhydrous/anhydrate crystal form of Form I has essentially no water content, showing no significant weight loss (less than 1.0%, 0.5% or 0.1% w/w) by TGA (FIG. 2) in the DSC (FIG. 1). DVS data (see FIG. 3) of Table 2 reveal little weight gain for Form I, indicating that it is substantially non-hygroscopic.

TABLE 2

| | Form 1 |
|---|---|
| TGA | No significant weight loss (less than 1.0%, |
| | 0.5%, or 0.1% w/w) before melting event |
| DSC | Melting onsets ~92° C. and ~99° C. |
| DVS | 0-60% RH, 25° C.: <0.1% weight gain |
| | 60% RH, 25° C.: <0.1% weight gain |
| | 75% RH, 25° C.: <0.1% weight gain |
| | 90% RH, 25° C.: <0.1% weight gain |

The crystal form of Form I can also be identified by its characteristic differential scanning (DSC) trace such as shown in FIG. 1. In some embodiments, Form I exhibits a differential scanning calorimetry trace comprising a melting endotherm having an onset at 92±5° C. and a melting endotherm having an onset at 99±5° C. Not wishing to be bound to any particular theory, it is believed the melting endotherm having an onset at 92±5° C. corresponds to Form I's melting point, while the melting endotherm having an onset at 99±5° C. may correspond to another solid form of the compound. In some embodiments, Form I exhibits a differential scanning calorimetry trace substantially lacking an endotherm corresponding to a dehydration event. In some further embodiments, Form I exhibits a DSC trace substantially as shown in FIG. 1. For DSC, it is known that the temperatures observed will depend upon the rate of temperature change as well as sample preparation technique and the particular instrument employed. Thus, the values reported herein relating to DSC thermograms can vary by plus or minus about 5° C.

In some embodiments, Form I can have a thermogravimetric analysis profile showing less than about 1.2%, less than about 1.0%, less than about 0.8%, less than about 0.5%, less than about 0.3%, less than about 0.2%, or less than about 0.1% weight loss from about 30° C. to about 90° C. In some further embodiments, the crystal form can have a have a thermogravimetric analysis profile substantially as shown in FIG. 2.

In some embodiments, the present invention provides a compound that is 1,1,1,3,3,3-hexafluoropropan-2-yl (3R)-3-[(cyclopropylsulfonyl)amino]-1-oxa-8-azaspiro[4.5]decane-8-carboxylate, or a pharmaceutically acceptable salt thereof. In some embodiments, the present invention provides the compound of 1,1,1,3,3,3-hexafluoropropan-2-yl (3R)-3-[(cyclopropylsulfonyl)amino]-1-oxa-8-azaspiro[4.5]decane-8-carboxylate. In some embodiments, the present invention provides a pharmaceutically acceptable salt of 1,1,1,3,3,3-hexafluoropropan-2-yl (3R)-3-[(cyclopropylsulfonyl)amino]-1-oxa-8-azaspiro[4.5]decane-8-carboxylate.

In some embodiments, the present invention provides a crystal form of anhydrous (anhydrate) 1,1,1,3,3,3-hexafluoropropan-2-yl (3R)-3-[(cyclopropylsulfonyl)amino]-1-oxa-8-azaspiro[4.5]decane-8-carboxylate. In some further embodiments, the crystal form of anhydrous (an hydrate) 1,1,1,3,3,3-hexafluoropropan-2-yl (3R)-3-[(cyclopropylsulfonyl)amino]-1-oxa-8-azaspiro[4.5]decane-8-carboxylate is Form A, that is characterized according to its unique solid state signatures with respect to, for example, powder X-ray diffraction (PXRD), differential scanning calorimetry (DSC), and/or other solid state methods. In some embodiments, the anhydrous crystal form of the invention (Form A) is substantially free of water or other organic solvent. Further characterization with respect to water or solvent content of the crystal forms can be gauged by any of various routine methods such as thermogravimetric analysis (TGA), dynamic vapor sorption (DVS), DSC and other techniques.

In some embodiments, the present invention provides a crystal form of anhydrous (anhydrate) 1,1,1,3,3,3-hexafluoropropan-2-yl (3R)-3-[(cyclopropylsulfonyl)amino]-1-oxa-8-azaspiro[4.5]decane-8-carboxylate, designated herein as “Form A,” having a powder X-ray diffraction pattern substantially as depicted in FIG. 8. A list of diffraction peaks expressed in terms of the degree 2θ and relative intensities with a relative intensity of ≥4.0% is provided above in Table 3.

TABLE 3

| Peak position (2 theta) | Relative Intensity (%) |
|---|---|
| 4.8 | 20.9 |
| 9.6 | 100 |
| 13.6 | 6.2 |
| 13.7 | 4.1 |
| 14.5 | 15.5 |
| 14.6 | 4.1 |
| 17.0 | 13.3 |
| 17.4 | 35.5 |
| 18.0 | 11.5 |
| 18.2 | 5.9 |
| 19.2 | 5.5 |
| 19.3 | 7.3 |
| 20.9 | 5.0 |
| 21.6 | 5.8 |
| 22.2 | 12.4 |
| 24.2 | 8.0 |
| 25.2 | 5.3 |
| 25.8 | 4.3 |
| 28.6 | 6.0 |
| 34.2 | 5.3 |

In some embodiments, Form A exhibits a powder X-ray diffraction pattern comprising at least two peaks, in terms of 2θ, selected from at 4.8±0.2°; 9.6±0.2°; 14.5±0.2°; 17.0±0.2°; and 17.4±0.2°. In some embodiments, Form A exhibits a powder X-ray diffraction pattern comprising at least three peaks, in terms of 2θ, selected from at 4.8±0.2°; 9.6±0.2°; 14.5±0.2°; 17.0±0.2°; and 17.4±0.2°. In some embodiments, Form A exhibits a powder X-ray diffraction pattern comprising at least four peaks, in terms of 2θ, selected from at 4.8±0.2°; 9.6±0.2°; 14.5±0.2°; 17.0±0.2°; and 17.4±0.2°.

In some embodiments, Form A exhibits a powder X-ray diffraction pattern comprising characteristic peaks, in terms of 2θ, at 4.8±0.2° and 9.6±0.2°.

In some embodiments, Form A exhibits a powder X-ray diffraction pattern comprising peaks, in terms of 2θ, at 4.8±0.2°; 9.6±0.2°; and 17.4±0.2°. In some further embodiments, Form A exhibits the X-ray powder diffraction pattern further comprises at least one peak, in terms of 2θ, selected from at 14.5±0.2°; and 17.0±0.2°.

In some embodiments, Form A exhibits a powder X-ray diffraction pattern comprising peaks, in terms of 2θ, at 4.8±0.2°; 9.6±0.2°; 14.5±0.2°; and 17.4±0.2°.

In some embodiments, Form A exhibits a powder X-ray diffraction pattern comprising peaks, in terms of 2θ, at 4.8±0.2°; 9.6±0.2°; 14.5±0.2°; 17.0±0.2°; and 17.4±0.2°. In some further embodiments, Form A exhibits the X-ray powder diffraction pattern further comprises at least one peak, in terms of 2θ, selected from at 13.6±0.2°; 13.7±0.2°; 18.0±0.2°; 18.2±0.2°; and 22.2±0.2°.

In some embodiments, Form A exhibits a powder X-ray diffraction pattern substantially as shown in FIG. 8.

As is well known in the art of powder diffraction, the relative intensities of the peaks (reflections) can vary, depending upon the sample preparation technique, the sample mounting procedure and the particular instrument employed. Moreover, instrument variation and other factors can affect the 2-theta values. Therefore, the XRPD peak assignments can vary by plus or minus about 0.2°.

Data of Table 4 pertaining to water content of the crystal form of Form A, shows that the anhydrous/anhydrate crystal form of Form A has essentially no water content, showing less than 0.5%, 0.2% or 0.1% weight loss by TGA (FIG. 6) in the DSC (FIG. 5). DVS data (see FIG. 7) of Table 2 reveal little weight gain for Form A, indicating that it is substantially non-hygroscopic.

TABLE 4

| | Form A |
|---|---|
| TGA | No significant weight loss (about less than |
| | 0.5%, 0.2% or 0.1%) before the melting |
| | event |
| DSC | Melting onset ~96° C. |
| DVS | 0-60% RH, 25° C.: <0.1% weight gain |
| | 60% RH, 25° C.: <0.1% weight gain |
| | 75% RH, 25° C.: <0.1% weight gain |
| | 90% RH, 25° C.: <0.1% weight gain |

The crystal form of Form A can also be identified by its characteristic differential scanning (DSC) trace such as shown in FIG. 5. In some embodiments, Form A exhibits a differential scanning calorimetry trace comprising a melting endotherm having an onset at 96±5° C. In some embodiments, Form I exhibits a differential scanning calorimetry trace substantially lacking an endotherm corresponding to a dehydration event. In some further embodiments, Form I exhibits a DSC trace substantially as shown in FIG. 5. For DSC, it is known that the temperatures observed will depend upon the rate of temperature change as well as sample preparation technique and the particular instrument employed. Thus, the values reported herein relating to DSC thermograms can vary by plus or minus about 5° C.

In some embodiments, Form A can have a thermogravimetric analysis profile showing less than about 0.5%, less than about 0.4%, less than about 0.3%, less than about 0.2%, or less than about 0.1% weight loss from about 30° C. to about 95° C. In some further embodiments, the crystal form can have a have a thermogravimetric analysis profile substantially as shown in FIG. 6.

In some embodiments, the present invention provides a compound that is 1,1,1,3,3,3-hexafluoropropan-2-yl 3-{[(cyclopropylmethyl)sulfonyl]amino}-1-oxa-9-azaspiro[5.5]undecane-9-carboxylate, or a pharmaceutically acceptable salt thereof. In some embodiments, the present invention provides the compound of 1,1,1,3,3,3-hexafluoropropan-2-yl 3-{[(cyclopropylmethyl)sulfonyl]amino}-1-oxa-9-azaspiro[5.5]undecane-9-carboxylate. In some embodiments, the present invention provides a pharmaceutically acceptable salt of 1,1,1,3,3,3-hexafluoropropan-2-yl 3-{[(cyclopropylmethyl)sulfonyl]amino}-1-oxa-9-azaspiro[5.5]undecane-9-carboxylate.

In some embodiments, the present invention provides a compound that is 1,1,1,3,3,3-hexafluoropropan-2-yl 3-{[(cyclopropylmethyl)sulfonyl]amino}-1-oxa-9-azaspiro[5.5]undecane-9-carboxylate, ENT-1, or a pharmaceutically acceptable salt thereof. In some embodiments, the present invention provides the compound of 1,1,1,3,3,3-hexafluoropropan-2-yl 3-{[(cyclopropylmethyl)sulfonyl]amino}-1-oxa-9-azaspiro[5.5]undecane-9-carboxylate, ENT-1. In some embodiments, the present invention provides a pharmaceutically acceptable salt of 1,1,1,3,3,3-hexafluoropropan-2-yl 3-{[(cyclopropylmethyl)sulfonyl]amino}-1-oxa-9-azaspiro[5.5]undecane-9-carboxylate, ENT-1.

In some embodiments, the present invention provides a compound that is 1,1,1,3,3,3-hexafluoropropan-2-yl 3-{[(cyclopropylmethyl)sulfonyl]amino}-1-oxa-9-azaspiro[5.5]undecane-9-carboxylate, ENT-2, or a pharmaceutically acceptable salt thereof. In some embodiments, the present invention provides the compound of 1,1,1,3,3,3-hexafluoropropan-2-yl 3-{[(cyclopropylmethyl)sulfonyl]amino}-1-oxa-9-azaspiro[5.5]undecane-9-carboxylate, ENT-2. In some embodiments, the present invention provides a pharmaceutically acceptable salt of 1,1,1,3,3,3-hexafluoropropan-2-yl 3-{[(cyclopropylmethyl)sulfonyl]amino}-1-oxa-9-azaspiro[5.5]undecane-9-carboxylate, ENT-1.

In some embodiments, the present invention provides a compound selected from Examples 1 to 5 in the EXAMPLES section or a pharmaceutically acceptable salt thereof (or the parent compound thereof where the exemplary compound, for example, is a salt) herein below.

The present invention includes any subset of any embodiment described herein.

The present invention includes combinations of two or more embodiments described hereinabove, or any subset thereof.

The present invention further provides the compound of the invention or a pharmaceutically acceptable salt thereof (including all embodiments and combinations of two or more embodiments described herein or any subcombination thereof) for use in the treatment of a MAGL-mediated disease or disorder described herein.

The present invention further provides use of the compound of the invention or a pharmaceutically acceptable salt thereof (including all embodiments and combinations of two or more embodiments described herein or any subcombination thereof) for treating a MAGL-mediated disease or disorder disorder described herein.

The present invention further provides a method for treating a MAGL-mediated disease or disorder in a patient (e.g., a mammal such as a human) comprising administering to the patient a therapeutically effective amount of the compound of the invention or a pharmaceutically acceptable salt thereof (including all embodiments and combinations of two or more embodiments described herein or any subcombination thereof).

The present invention further provides use of the compound of the invention or a pharmaceutically acceptable salt thereof (including all embodiments and combinations of two or more embodiments described herein or any subcombination thereof) in the manufacture of a medicament for use in the treatment of a MAGL-mediated disease or disorder described herein.

The compound of the invention or a pharmaceutically acceptable salt thereof of the present invention (or a metabolite thereof) is a MAGL inhibitor. Thus, the present invention further provides a method for inhibiting MAGL (i.e., an activity of MAGL either in vitro or in vivo), comprising contacting (including incubating) the MAGL with the compound of the invention or a pharmaceutically acceptable salt thereof (such as one selected from Examples 1-5 herein) described herein.

As used herein, the term “contacting” refers to the bringing together of indicated moieties in an in vitro system or an in vivo system. For example, “contacting” MAGL with a compound of the invention includes the administration of a compound of the present invention to an individual or patient, such as a human, having the MAGL, as well as, for example, introducing a compound of the invention into a sample containing a cellular or purified preparation containing the MAGL.

The amount of the compound of the invention or a pharmaceutically acceptable salt thereof used in any one of the methods (or uses) of the present invention is effective in inhibiting MAGL.

MAGL-mediated diseases or disorders include, for example, a metabolic disorder (e.g., obesity); vomiting or emesis; nausea; an eating disorder (e.g anorexia or bulimia); neuropathy (e.g., diabetic neuropathy, pellagric neuropathy, alcoholic neuropathy, Beriberi neuropathy); burning feet syndrome; a neurodegenerative disorder [multiple sclerosis (MS), Parkinson's disease (PD), Huntington's disease, Alzheimer's disease, amyotrophic lateral sclerosis (ALS), epilepsy, a sleep disorder, Creutzfeldt-Jakob disease (CJD), or prion disease]; a cardiovascular disease (e.g., hypertension, dyslipidemia, atherosclerosis, cardiac arrhythmias, or cardiac ischemia); osteoporosis; osteoarthritis; schizophrenia; depression; bipolar disease; tremor; dyskinesia; dystonia; spasticity; Tourette's syndrome; sleep apnea; hearing loss; an eye disease (e.g., glaucoma, ocular hypertension, macular degeneration, or a disease arising from elevated intraocular pressure); cachexia; insomnia; meningitis; sleeping sickness; progressive multifocal leukoencephalopathy; De Vivo disease; cerebral edema; cerebral palsy; withdrawal syndrome [alcohol withdrawal syndrome, antidepressant discontinuation syndrome, antipsychotic withdrawal syndrome, benzodiazepine withdrawal syndrome, cannabis withdrawal, neonatal withdrawal, nicotine withdrawal, or opioid withdrawal]; traumatic brain injury; spinal cord injury; seizures; excitotoxin exposure; ischemia [stroke, hepatic ischemia or reperfusion, CNS ischemia or reperfusion]; liver fibrosis, iron overload, cirrhosis of the liver; a lung disorder [asthma, allergies, COPD, chronic bronchitis, emphysema, cystic fibrosis, pneumonia, tuberculosis, pulmonary edema, lung cancers, acute respiratory distress syndrome, intersitital lung disease (ILD), sarcoidosis, idiopathic pulmonary fibrosis, pulmonary embolism, pleural effusion, or mesothelioma]; a liver disorder [acute liver failure, Alagille syndrome, hepatitis, enlarged liver, Gilbert's syndrome, liver cysts, liver hemangioma, fatty liver disease, steatohepatitis [e.g. nonalcoholic Steatohepatitis (NASH)], primary sclerosing cholangitis, fascioliasis, primary bilary cirrhosis, Budd-Chiari syndrome, hemochromatosis, Wilson's disease, or transthyretin-related hereditary amyloidosis], stroke [e.g., ischemic stroke; hemorrhagic stroke]; subarachnoid hemorrhage; vasospasm; AIDS wasting syndrome; renal ischemia; a disorder associated with abnormal cell growth or proliferation [e.g., a benign tumor or cancer such as benign skin tumor, brain tumor, papilloma, prostate tumor, cerebral tumor (glioblastoma, medulloepithelioma, medulloblastoma, neuroblastoma, astrocytoma, astroblastoma, ependymoma, oligodendroglioma, plexus tumor, neuroepithelioma, epiphyseal tumor, ependymoblastoma, malignant meningioma, sarcomatosis, melanoma, schwannoma), melanoma, metastatic tumor, kidney cancer, bladder cancer, brain cancer, glioblastoma (GBM), gastrointestinal cancer, leukemia or blood cancer]; an autoimmune disease [e.g., psoriasis, lupus erythematosus, Sjogren's syndrome, ankylosing spondylitis, undifferentiated spondylitis, Behcet's disease, hemolytic anemia, graft rejection]; an inflammatory disorder [e.g., appendicitis, bursitis, colitis, cystitis, dermatitis, phlebitis, rhinitis, tendonitis, tonsillitis, vasculitis, acne vulgaris, chronic prostatitis, glomerulonephritis, hypersensitivities, IBS, pelvic inflammatory disease, sarcoidosis, HIV encephalitis, rabies, brain abscess, neuroinflammation, inflammation in the central nervous system (CNS)]; a disorder of the immune system (e.g., transplant rejection or celiac disease); post-traumatic stress disorder (PTSD); acute stress disorder; panic disorder; substance-induced anxiety; obsessive-compulsive disorder (OCD); agoraphobia; specific phobia; social phobia; anxiety disorder; attention deficit disorder (ADD); attention deficit hyperactivity disorder (ADHD); Asperger's syndrome; pain [e.g., acute pain; chronic pain; inflammatory pain; visceral pain; post-operative pain; migraine; lower back pain; joint pain; abdominal pain; chest pain; postmastectomy pain syndrome; menstrual pain; endometriosis pain; pain due to physical trauma; headache; sinus headache; tension headache arachnoiditis, herpes virus pain, diabetic pain; pain due to a disorder selected from: osteoarthritis, rheumatoid arthritis, osteoarthritis, spondylitis, gout, labor, musculoskeletal disease, skin disease, toothache, pyresis, burn, sunburn, snake bite, venomous snake bite, spider bite, insect sting, neurogenic bladder, interstitial cystitis, urinary tract infection (UTI), rhinitis, contact dermatitis/hypersensitivity, itch, eczema, pharyngitis, mucositis, enteritis, irritable bowel syndrome (IBS), cholecystitis, and pancreatitis; neuropathic pain (e.g., neuropathic low back pain, complex regional pain syndrome, post trigeminal neuralgia, causalgia, toxic neuropathy, reflex sympathetic dystrophy, diabetic neuropathy, chronic neuropathy from chemotherapeutic agent, or sciatica pain)]; a demyelinating disease [e.g., multiple sclerosis (MS), Devic's disease, CNS neuropathies, central pontine myelinolysis, syphilitic myelopathy, leukoencephalopathies, leukodystrophies, Guillain-Barre syndrome, chronic inflammatory demyelinating polyneuropathy, anti-myelin-associated glycoprotein (MAG) peripheral neuropathy, Charcot-Marie-Tooth disease, peripheral neuropathy, myelopathy, optic neuropathy, progressive inflammatory neuropathy, optic neuritis, transverse myelitis]; and cognitive impairment [e.g., cognitive impairment associated with Down's syndrome; cognitive impairment associated with Alzheimer's disease; cognitive impairment associated with PD; mild cognitive impairment (MCI), dementia, post-chemotherapy cognitive impairment (PCCI), postoperative cognitive dysfunction (POCD)].

The term “therapeutically effective amount” as used herein refers to that amount of the compound (including a pharmaceutically acceptable salt thereof) being administered which will relieve to some extent one or more of the symptoms of the disorder being treated. In reference to the treatment of a MAGL-mediated disease or disorder (e.g., Alzheimer's disease, inflammation, or pain), a therapeutically effective amount refers to that amount which has the effect of relieving to some extent (or, for example, eliminating) one or more symptoms associated with the MAGL-mediated disease or disorder (e.g., psychotic symptom of Alzheimer's disease).

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined herein. The term “treating” also includes adjuvant and neo-adjuvant treatment of a subject.

As noted above, the compounds of the invention may exist in the form of pharmaceutically acceptable salts such as acid addition salts and/or base addition salts of the compounds of the invention. The phrase “pharmaceutically acceptable salt(s)”, as used herein, unless otherwise indicated, includes acid addition or base salts which may be present in the compounds of the invention.

Pharmaceutically acceptable salts of the compounds of the invention include the acid addition and base salts thereof.

Suitable acid addition salts are formed from acids which form non-toxic salts. Examples include the acetate, adipate, aspartate, benzoate, besylate, bicarbonate/carbonate, bisulfate/sulfate, borate, camphorsulfonate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see “Handbook of Pharmaceutical Salts: Properties, Selection, and Use” by Stahl and Wermuth (Wiley-VCH, 2002). Methods for making pharmaceutically acceptable salts of compounds of the invention are known to one of skill in the art.

As used herein the terms “the compound of invention” or “the compound of invention or a pharmaceutically acceptable salt thereof” are defined to include all forms of the compound of the invention or pharmaceutically salt thereof, including anhydrates (anhydrous forms), hydrates, solvates, isomers (including for example rotational stereoisomers), crystalline and non-crystalline forms, isomorphs, polymorphs, metabolites, and prodrugs thereof.

As is known to the person skilled in the art, amine compounds (i.e., those comprising one or more nitrogen atoms), for example tertiary amines, can form N-oxides (also known as amine oxides or amine N-oxides). An N-oxide has the formula of (R¹⁰⁰)(R²⁰⁰)(R³⁰⁰)N⁺—O⁻ wherein the parent amine (R¹⁰⁰)(R²⁰⁰)(R³⁰⁰)N can be, for example, a tertiary amine (for example, each of R¹⁰⁰, R²⁰⁰, R³⁰⁰ is independently alkyl, arylalkyl, aryl, heteroaryl, or the like), a heterocyclic or heteroaromatic amine [for example, (R¹⁰⁰)(R²⁰⁰)(R³⁰⁰)N together forms 1-alkylpiperidine, 1-alkylpyrrolidine, 1-benzylpyrrolidine, or pyridine]. For instance, an imine nitrogen, especially a heterocyclic or heteroaromatic imine nitrogen, or pyridine-type nitrogen

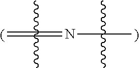

atom [such as a nitrogen atom in pyridine, pyridazine, or pyrazine], can be N-oxidized to form the N-oxide comprising the group

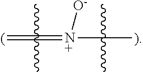

Thus, a compound according to the present invention comprising one or more nitrogen atoms (e.g., an imine nitrogen atom) may be capable of forming an N-oxide thereof (e.g., mono-N-oxides, bis-N-oxides or multi-N-oxides, or mixtures thereof depending on the number of nitrogen atoms suitable to form stable N-oxides).

As used herein, the term “N-oxide(s)” refer to all possible, and in particular all stable, N-oxide forms of the amine compounds (e.g., compounds comprising one or more imine nitrogen atoms) described herein, such as mono-N-oxides (including different isomers when more than one nitrogen atom of an amine compound can form a mono-N-oxide) or multi-N-oxides (e.g., bis-N-oxides), or mixtures thereof in any ratio.

Compounds of the invention and their salts described herein further include N-oxides thereof.

In the description herein below, unless otherwise specified, compounds of the invention (or compounds of the invention) include salts of the compounds and the N-oxides of the compounds or the salts.

As is also known to the person skilled in the art, tertiary amine compounds (i.e., those comprising one or more tertiary amine nitrogen atoms) can form quaternary ammonium salts. In the description herein below, unless otherwise specified, compounds of the invention (or compounds of the invention) further include their quaternary ammonium salts.

Compounds of the invention may exist in a continuum of solid states ranging from fully amorphous to fully crystalline. The term ‘amorphous’ refers to a state in which the material lacks long-range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from apparent solid to a material with liquid properties occurs, which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

Compounds of the invention may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may exist as clathrates or other complexes (e.g., co-crystals). Included within the scope of the invention are complexes such as clathrates, drug-host inclusion complexes wherein the drug and host are present in stoichiometric or non-stoichiometric amounts. Also included are complexes of the compounds of the invention containing two or more organic and/or inorganic components, which may be in stoichiometric or non-stoichiometric amounts. The resulting complexes may be ionized, partially ionized, or non-ionized. Co-crystals are typically defined as crystalline complexes of neutral molecular constituents that are bound together through non-covalent interactions, but could also be a complex of a neutral molecule with a salt. Co-crystals may be prepared by melt crystallization, by recrystallization from solvents, or by physically grinding the components together; see O. Almarsson and M. J. Zaworotko, Chem. Commun. 2004, 17, 1889-1896. For a general review of multi-component complexes, see J. K. Haleblian, J. Pharm. Sci. 1975, 64, 1269-1288.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

The invention also relates to prodrugs of the compounds of the invention. Thus certain derivatives of compounds of the invention which may have little or no pharmacological activity themselves can, when administered into or onto the body, be converted into compounds of the invention having the desired activity, for example, by hydrolytic cleavage. Such derivatives are referred to as “prodrugs”. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of the invention with certain moieties known to those skilled in the art as ‘pro-moieties’ as described, for example, in Design of Prodrugs by H. Bundgaard (Elsevier, 1985), or in Prodrugs: Challenges and Reward, 2007 edition, edited by Valentino Stella, Ronald Borchardt, Michael Hageman, Reza Oliyai, Hans Maag, Jefferson Tilley, pages 134-175 (Springer, 2007).

Moreover, certain compounds of the invention may themselves act as prodrugs of other compounds of the invention.

Also included within the scope of the invention are metabolites of compounds of the invention, that is, compounds formed in vivo upon administration of the drug.

The compounds of the invention include all stereoisomers and tautomers. Stereoisomers of the invention include cis and trans isomers, optical isomers such as R and S enantiomers, diastereomers, geometric isomers, rotational isomers, atropisomers, and conformational isomers of the compounds of the invention, including compounds exhibiting more than one type of isomerism; and mixtures thereof (such as racemates and diastereomeric pairs). Also included are acid addition or base addition salts wherein the counterion is optically active, for example, D-lactate or L-lysine, or racemic, for example, DL-tartrate or DL-arginine.

In some embodiments, the compounds of the invention (including salts thereof) may have asymmetric carbon atoms. The carbon-carbon bonds of the compounds of the invention may be depicted herein using a solid line ( [symbol] ) a wavy line ( [symbol] ), a solid wedge ( [symbol] ), or a dotted wedge ( [symbol] ). The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. The use of a wavy line to depict bonds to asymmetric carbon atoms is meant to indicate that the stereochemistry is unknown (unless otherwise specified). It is possible that compounds of the invention may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of the invention can exist as enantiomers and diastereomers or as racemates and mixtures thereof. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

In some embodiments, the compounds of the invention may exist in and/or be isolated as atropisomers (e.g., one or more atropenantiomers). Those skilled in the art would recognize that atropisomerism may exist in a compound that has two or more aromatic rings (for example, two aromatic rings linked through a single bond). See e.g., Freedman, T. B. et al., Absolute Configuration Determination of Chiral Molecules in the Solution State Using Vibrational Circular Dichroism. Chirality 2003, 15, 743-758; and Bringmann, G. et al., Atroposelective Synthesis of Axially Chiral Biaryl Compounds. Angew. Chem., Int. Ed. 2005, 44, 5384-5427.

When any racemate crystallizes, crystals of different types are possible. One type is the racemic compound (true racemate) wherein one homogeneous form of crystal is produced containing both enantiomers in equimolar amounts. Another type is a racemic mixture or conglomerate wherein two forms of crystal are produced in equal or different molar amounts each comprising a single enantiomer.

The compounds of the invention may exhibit the phenomena of tautomerism and structural isomerism. For example, the compounds of the invention may exist in several tautomeric forms, including the enol and imine form, the amide and imidic acid form, and the keto and enamine form and geometric isomers and mixtures thereof. All such tautomeric forms are included within the scope of the compounds of the invention. Tautomers may exist as mixtures of a tautomeric set in solution. In solid form, usually one tautomer predominates. Even though one tautomer may be described, the present invention includes all tautomers of the compounds of the invention. For example, when one of the following two tautomers (wherein R can be, for example, phenyl that is further substituted) is disclosed, those skilled in the art would readily recognize the other tautomer.

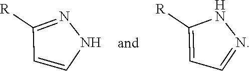

The present invention includes all pharmaceutically acceptable isotopically labelled compounds of the invention or salts thereof wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically labelled compounds of the invention, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron-emitting isotopes, such as ¹¹C, ¹⁸F, ¹⁵O and ¹³N, can be useful in Positron Emission Topography (PET) studies for examining substrate receptor occupancy.

Isotopically labeled compounds of the invention can generally be prepared by conventional techniques known to those skilled in the art or by processes analogous to those described in the accompanying Examples and Preparations using an appropriate isotopically labeled reagent in place of the non-labeled reagent previously employed.

The present invention also provides compositions (e.g., pharmaceutical compositions) comprising a novel compound of the invention. Accordingly, in one embodiment, the invention provides a pharmaceutical composition comprising (a therapeutically effective amount of) a novel compound of the invention or a pharmaceutically acceptable salt thereof and optionally comprising a pharmaceutically acceptable carrier. In one further embodiment, the invention provides a pharmaceutical composition comprising (a therapeutically effective amount of) a compound of the invention or a pharmaceutically acceptable salt thereof, optionally comprising a pharmaceutically acceptable carrier and, optionally, at least one additional medicinal or pharmaceutical agent (such as an antipsychotic agent or anti-schizophrenia agent described below). In one embodiment, the additional medicinal or pharmaceutical agent is an anti-schizophrenia agent as described below.

The pharmaceutically acceptable carrier may comprise any conventional pharmaceutical carrier or excipient. Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus for oral administration, tablets containing various excipients, such as citric acid, may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration, the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulation, solution or suspension, for parenteral injection as a sterile solution, suspension or emulsion, for topical administration as an ointment or cream or for rectal administration as a suppository.

Exemplary parenteral administration forms include solutions or suspensions of active compounds in sterile aqueous solutions, for example, aqueous propylene glycol or dextrose solutions. Such dosage forms may be suitably buffered, if desired.

The pharmaceutical composition may be in unit dosage forms suitable for single administration of precise dosages. One of ordinary skill in the art would appreciate that the composition may be formulated in sub-therapeutic dosage such that multiple doses are envisioned.

In one embodiment the composition comprises a therapeutically effective amount of a compound of the invention or salt thereof and a pharmaceutically acceptable carrier.

Compounds of the invention (including salts thereof) are MAGL inhibitors. In some embodiments, the IC₅₀ of a compound of the invention (or its metabolite) is less than about 10 μM, 5 μM, 2 μM, 1 μM, 500 nM, 200 nM, 100 nM, 50, 40, 30, 20, 10, 5, 2, or 1 nM as determined by the method in Example AA described herein below.

Administration of the compounds of the invention (including salts thereof) may be effected by any method that enables delivery of the compounds to the site of action. These methods include, for example, enteral routes (e.g., oral routes, buccal routes, sublabial routes, sublingual routes), oral routes, intranasal routes, inhaled routes, intraduodenal routes, parenteral injection (including intravenous, subcutaneous, intramuscular, intravascular or infusion), intrathecal routes, epidural routes, intracerebral routes, intracerbroventricular routes, topical, and rectal administration.

In one embodiment of the present invention, the compounds of the invention may be administered/effected by parenteral injection routes (e.g., intravenous injection route).

In one embodiment of the present invention, the compounds of the invention may be administered/effected by oral routes.

Dosage regimens may be adjusted to provide the optimum desired response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specifications for the dosage unit forms of the invention are dictated by a variety of factors such as the unique characteristics of the therapeutic agent and the particular therapeutic or prophylactic effect to be achieved. In one embodiment of the present invention, the compounds of the invention may be used to treat humans.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent is well-known in the relevant art and would be understood to be encompassed by the skilled artisan once provided the teachings disclosed herein.

The amount of the compound of the invention administered will be dependent on the subject being treated, the severity of the disorder or condition, the rate of administration, the disposition of the compound and the discretion of the prescribing physician. Generally, an effective dosage is in the range of about 0.0001 to about 50 mg per kg body weight per day, for example about 0.01 to about 10 mg/kg/day, in single or divided doses. For a 70 kg human, this would amount to about 0.007 mg to about 3500 mg/day, for example about 0.7 mg to about 700 mg/day. In some instances, dosage levels below the lower limit of the aforesaid range may be more than adequate, while in other cases still larger doses may be employed without causing any harmful side effect, provided that such larger doses are first divided into several small doses for administration throughout the day.

As used herein, the term “combination therapy” refers to the administration of a compound of the invention or a pharmaceutically acceptable salt thereof together with an at least one additional pharmaceutical or medicinal agent (e.g., an anti-schizophrenia agent), either sequentially or simultaneously.

The present invention includes the use of a combination of a compound of the invention (including a salt thereof) and one or more additional pharmaceutically active agent(s). If a combination of active agents is administered, then they may be administered sequentially or simultaneously, in separate dosage forms or combined in a single dosage form. Accordingly, the present invention also includes pharmaceutical compositions comprising an amount of: (a) a first agent comprising a compound of the invention (including a pharmaceutically acceptable salt thereof); (b) a second pharmaceutically active agent; and (c) a pharmaceutically acceptable carrier, vehicle or diluent.

Various pharmaceutically active agents may be selected for use in conjunction with the compounds of the invention, depending on the disease, disorder, or condition to be treated. Pharmaceutically active agents that may be used in combination with the compositions of the present invention include, without limitation:

(i) acetylcholinesterase inhibitors such as donepezil hydrochloride (ARICEPT, MEMAC); or Adenosine A_{2A} receptor antagonists such as Preladenant (SCH 420814) or SCH 412348;
(ii) amyloid-ß (or fragments thereof), such as Aß₁₋₁₅ conjugated to pan HLA DR-binding epitope (PADRE) and ACC-001 (Elan/Wyeth);
(iii) antibodies to amyloid-ß (or fragments thereof), such as bapineuzumab (also known as AAB-001) and AAB-002 (Wyeth/Elan);
(iv) amyloid-lowering or -inhibiting agents (including those that reduce amyloid production, accumulation and fibrillization) such as colostrinin and bisnorcymserine (also known as BNC);
(v) alpha-adrenergic receptor agonists such as clonidine (CATAPRES);
(vi) beta-adrenergic receptor blocking agents (beta blockers) such as carteolol;
(vii) anticholinergics such as amitriptyline (ELAVIL, ENDEP);
(viii) anticonvulsants such as carbamazepine (TEGRETOL, CARBATROL);
(ix) antipsychotics, such as lurasidone (also known as SM-13496; Dainippon Sumitomo);
(x) calcium channel blockers such as nilvadipine (ESCOR, NIVADIL);
(xi) catechol O-methyltransferase (COMT) inhibitors such as tolcapone (TASMAR);
(xii) central nervous system stimulants such as caffeine;
(xiii) corticosteroids such as prednisone (STERAPRED, DELTASONE);
(xiv) dopamine receptor agonists such as apomorphine (APOKYN);
(xv) dopamine receptor antagonists such as tetrabenazine (NITOMAN, XENAZINE, dopamine D2 antagonist such as Quetiapine);
(xvi) dopamine reuptake inhibitors such as nomifensine maleate (MERITAL);
(xvii) gamma-aminobutyric acid (GABA) receptor agonists such as baclofen (LIORESAL, KEMSTRO);
(xviii) histamine 3 (H₃) antagonists such as ciproxifan;
(xix) immunomodulators such as glatiramer acetate (also known as copolymer-1; COPAXONE);
(xx) immunosuppressants such as methotrexate (TREXALL, RHEUMATREX);
(xxi) interferons, including interferon beta-1a (AVONEX, REBIF) and interferon beta-1b (BETASERON, BETAFERON);
(xxii) levodopa (or its methyl or ethyl ester), alone or in combination with a DOPA decarboxylase inhibitor (e.g., carbidopa (SINEMET, CARBILEV, PARCOPA));
(xxiii) N-methyl-D-aspartate (NMDA) receptor antagonists such as memantine (NAMENDA, AXURA, EBIXA);
(xxiv) monoamine oxidase (MAO) inhibitors such as selegiline (EMSAM);
(xxv) muscarinic receptor (particularly M1 or M4 subtype) agonists such as bethanechol chloride (DUVOID, URECHOLINE);
(xxvi) neuroprotective drugs such as 2,3,4,9-tetrahydro-1H-carbazol-3-one oxime;
(xxvii) nicotinic receptor agonists such as epibatidine;
(xxviii) norepinephrine (noradrenaline) reuptake inhibitors such as atomoxetine (STRATTERA);
(xxix) phosphodiesterase (PDE) inhibitors, for example, PDE9 inhibitors such as BAY 73-6691 (Bayer AG) and PDE 10 (e.g., PDE10A) inhibitors such as papaverine;
(xxx) other PDE inhibitors including (a) PDE1 inhibitors (e.g., vinpocetine), (b) PDE2 inhibitors (e.g., erythro-9-(2-hydroxy-3-nonyl)adenine (EHNA)), (c) PDE4 inhibitors (e.g., rolipram), and (d) PDE5 inhibitors (e.g., sildenafil (VIAGRA, REVATIO));
(xxxi) quinolines such as quinine (including its hydrochloride, dihydrochloride, sulfate, bisulfate and gluconate salts);
(xxxii) β-secretase inhibitors such as WY-25105;
(xxxiii) γ-secretase inhibitors such as LY-411575 (Lilly);
(xxxiv) serotonin (5-hydroxytryptamine) 1A (5-HT_{1A}) receptor antagonists such as spiperone;
(xxxv) serotonin (5-hydroxytryptamine) 4 (5-HT₄) receptor agonists such as PRX-03140 (Epix);
(xxxvi) serotonin (5-hydroxytryptamine) 6 (5-HT₆) receptor antagonists such as mianserin (TORVOL, BOLVIDON, NORVAL);
(xxxvii) serotonin (5-HT) reuptake inhibitors such as alaproclate, citalopram (CELEXA, CIPRAMIL);
(xxxviii) trophic factors, such as nerve growth factor (NGF), basic fibroblast growth factor (bFGF; ERSOFERMIN), neurotrophin-3 (NT-3), cardiotrophin-1, brain-derived neurotrophic factor (BDNF), neublastin, meteorin, and glial-derived neurotrophic factor (GDNF), and agents that stimulate production of trophic factors, such as propentofylline;
(xxxix) antihemorrhagic (i.e., hemostatic) agents such as rivaroxaban or apixaban; and the like.

The compound of the invention (including a salt thereof) is optionally used in combination with another active agent. Such an active agent may be, for example, an atypical antipsychotic or an anti-Parkinson's disease agent or an anti-Alzheimer's agent. Accordingly, another embodiment of the invention provides methods of treating a MAGL-mediated disease or disorder in a mammal, comprising administering to the mammal an effective amount of a compound of the invention (including a pharmaceutically acceptable salt thereof) and further comprising administering another active agent.

As used herein, the term “another active agent” refers to any therapeutic agent, other than the compound of the invention (including or a pharmaceutically acceptable salt thereof) that is useful for the treatment of a subject disorder. Examples of additional therapeutic agents include antidepressants, antipsychotics (such as anti-schizophrenia), anti-pain, anti-Parkinson's disease agents, anti-LID (levodopa-induced dyskinesia), anti-Alzheimer's, anti-anxiety, and antihemorrhagic agents. Examples of particular classes of antidepressants that can be used in combination with the compounds of the invention include norepinephrine reuptake inhibitors, selective serotonin reuptake inhibitors (SSRIs), NK-1 receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Examples of suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Examples of suitable selective serotonin reuptake inhibitors include fluoxetine, fluvoxamine, paroxetine, and sertraline. Examples of monoamine oxidase inhibitors include isocarboxazid, phenelzine, and tranylcyclopramine. Examples of suitable reversible inhibitors of monoamine oxidase include moclobemide. Examples of suitable serotonin and noradrenaline reuptake inhibitors of use in the present invention include venlafaxine. Examples of suitable atypical antidepressants include bupropion, lithium, nefazodone, trazodone and viloxazine. Examples of anti-Alzheimer's agents include Dimebon, NMDA receptor antagonists such as memantine; and cholinesterase inhibitors such as donepezil and galantamine. Examples of suitable classes of anti-anxiety agents that can be used in combination with the compounds of the invention include benzodiazepines and serotonin 1A (5-HT1A) agonists or antagonists, especially 5-HT1A partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam. Suitable 5-HT1A receptor agonists or antagonists include buspirone, flesinoxan, gepirone, and ipsapirone. Suitable atypical antipsychotics include paliperidone, bifeprunox, ziprasidone, risperidone, aripiprazole, olanzapine, and quetiapine. Suitable nicotine acetylcholine agonists include ispronicline, varenicline and MEM 3454. Anti-pain agents include pregabalin, gabapentin, clonidine, neostigmine, baclofen, midazolam, ketamine and ziconotide. Examples of suitable anti-Parkinson's disease agents include L-DOPA (or its methyl or ethyl ester), a DOPA decarboxylase inhibitor (e.g., carbidopa (SINEMET, CARBILEV, PARCOPA), an Adenosine A_{2A} receptor antagonist [e.g., Preladenant (SCH 420814) or SCH 412348], benserazide (MADOPAR), a-methyldopa, monofluoromethyldopa, difluoromethyldopa, brocresine, or m-hydroxybenzylhydrazine), a dopamine agonist [such as apomorphine (APOKYN), bromocriptine (PARLODEL), cabergoline (DOSTINEX), dihydrexidine, dihydroergocryptine, fenoldopam (CORLOPAM), lisuride (DOPERGIN), pergolide (PERMAX), piribedil (TRIVASTAL, TRASTAL), pramipexole (MIRAPEX), quinpirole, ropinirole (REQUIP), rotigotine (NEUPRO), SKF-82958 (GlaxoSmithKline), and sarizotan], a monoamine oxidase (MAO) inhibitor [such as selegiline (EMSAM), selegiline hydrochloride (L-deprenyl, ELDEPRYL, ZELAPAR), dimethylselegilene, brofaromine, phenelzine (NARDIL), tranylcypromine (PARNATE), moclobemide (AURORIX, MANERIX), befloxatone, safinamide, isocarboxazid (MARPLAN), nialamide (NIAMID), rasagiline (AZILECT), iproniazide (MARSILID, IPROZID, IPRONID), CHF-3381 (Chiesi Farmaceutici), iproclozide, toloxatone (HUMORYL, PERENUM), bifemelane, desoxypeganine, harmine (also known as telepathine or banasterine), harmaline, linezolid (ZYVOX, ZYVOXID), and pargyline (EUDATIN, SUPIRDYL)], a catechol O-methyltransferase (COMT) inhibitor [such as tolcapone (TASMAR), entacapone (COMTAN), and tropolone], an N-methyl-D-aspartate (NMDA) receptor antagonist [such as amantadine (SYMMETREL)], anticholinergics [such as amitriptyline (ELAVIL, ENDEP), butriptyline, benztropine mesylate (COGENTIN), trihexyphenidyl (ARTANE), diphenhydramine (BENADRYL), orphenadrine (NORFLEX), hyoscyamine, atropine (ATROPEN), scopolamine (TRANSDERM-SCOP), scopolamine methylbromide (PARMINE), dicycloverine (BENTYL, BYCLOMINE, DIBENT, DILOMINE, tolterodine (DETROL), oxybutynin (DITROPAN, LYRINEL XL, OXYTROL), penthienate bromide, propantheline (PRO-BANTHINE), cyclizine, imipramine hydrochloride (TOFRANIL), imipramine maleate (SURMONTIL), lofepramine, desipramine (NORPRAMIN), doxepin (SINEQUAN, ZONALON), trimipramine (SURMONTIL), and glycopyrrolate (ROBINUL)], or a combination thereof. Examples of anti-schizophrenia agents include ziprasidone, risperidone, olanzapine, quetiapine, aripiprazole, asenapine, blonanserin, or iloperidone. Some additional “another active agent” examples include rivastigmine (Exelon), Clozapine, Levodopa, Rotigotine, Aricept, Methylphenidate, memantine. milnacipran, guanfacine, bupropion, and atomoxetine. Examples of antihemorrhagic agents (including, e.g., coagulation factors, activators, or stabilizers) include Factor Xa inhibitors (e.g., rivaroxaban or apixaban) and recombinant Coagulation Factor Vila (e.g., NovoSeven®).

As noted above, the compounds of the invention or salts thereof may be used in combination with one or more additional anti-Alzheimer's agents which are described herein. When a combination therapy is used, the one or more additional anti-Alzheimer's agents may be administered sequentially or simultaneously with the compound of the invention. In one embodiment, the additional anti-Alzheimer's agent(s) is(are) administered to a mammal (e.g., a human) prior to administration of the compound of the invention. In another embodiment, the additional anti-Alzheimer's agent(s) is(are) administered to the mammal after administration of the compound of the invention. In another embodiment, the additional anti-Alzheimer's agent(s) is(are) administered to the mammal (e.g., a human) simultaneously with the administration of the compound of the invention (or a pharmaceutically acceptable salt thereof).

The invention also provides a pharmaceutical composition for the treatment of an inflammatory disorder (e.g., nueroinflammation) in a mammal, including a human, which comprises an amount of a compound of the invention (including a salt thereof), as defined above (including hydrates, solvates and polymorphs of said compound or pharmaceutically acceptable salts thereof), in combination with one or more (for example one to three) anti-inflammation agents, wherein the amounts of the active agent and the combination when taken as a whole are therapeutically effective for treating the inflammatory disorder.

The invention also provides a pharmaceutical composition for treating a MAGL-mediated disease or disorder in a mammal, including a human, which comprises an amount of a compound of the invention (including a salt thereof), as defined above (including hydrates, solvates and polymorphs of said compound or a salt thereof), in combination with one or more (for example one to three) other agents for treating the MAGL-mediated disease or disorder, wherein the amount of the active agents and the combination when taken as a whole are therapeutically effective for treating the MAGL-mediated disease or disorder.

It will be understood that the compounds of the invention depicted above are not limited to a particular stereoisomer (e.g., enantiomer or diasteroisomer) shown, but also include all stereoisomers and mixtures thereof.

### BRIEF DESCRIPTION OF FIGURES

FIG. 1 represents a differential scanning calorimetry (DSC) thermogram of an anhydrous (anhydrate) crystal form (Form 1) of the compound of Example 1.

FIG. 2 represents a thermogravimetric analysis (TGA) of an anhydrous (anhydrate) crystal form (Form 1) of the compound of Example 1.

FIG. 3 depicts a dynamic vapor sorption (DVS) isotherm plot for an anhydrous (anhydrate) crystal form (Form 1) of the compound of Example 1.

FIG. 4 represents an observed powder X-ray diffraction pattern for an anhydrous (anhydrate) crystal form (Form 1) of the compound of Example 1.

FIG. 5 represents a differential scanning calorimetry (DSC) thermogram of an anhydrous (anhydrate) crystal form (Form A) of the compound of Example 2.

FIG. 6 represents a thermogravimetric analysis (TGA) of an anhydrous (anhydrate) crystal form (Form A) of the compound of Example 2.

FIG. 7 depicts a dynamic vapor sorption (DVS) isotherm plot for an anhydrous (anhydrate) crystal form (Form A) of the compound of Example 2.

FIG. 8 represents an observed powder X-ray diffraction pattern for an anhydrous (anhydrate) crystal form (Form A) of the compound of Example 2.

### DETAILED DESCRIPTION OF THE INVENTION

Compounds of the invention, including salts of the compounds, can be prepared using known organic synthesis techniques and can be synthesized according to any of numerous possible synthetic routes. The reactions for preparing compounds of the invention can be carried out in suitable solvents, which can be readily selected by one of skill in the art of organic synthesis. Suitable solvents can be substantially non-reactive with the starting materials (reactants), the intermediates, or products at the temperatures at which the reactions are carried out, e.g., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high-performance liquid chromatography (HPLC) or thin layer chromatography (TLC).

Compounds of the invention, salts and intermediates thereof may be prepared according to the reaction schemes described herein and accompanying discussion. In general, the compounds of this invention may be made by processes which include processes analogous to those known in the chemical arts, particularly in light of the description contained herein. Certain processes for the manufacture of the compounds of this invention and intermediates thereof are provided as further features of the invention and are illustrated by processes described in the experimental section. The schemes and examples provided herein (including the corresponding description) are for illustration only, and not intended to limit the scope of the present invention.

Compounds of the invention may exist as stereoisomers, such as atropisomers, racemates, enantiomers, or diastereomers. Conventional techniques for the preparation/isolation of individual enantiomers include chiral synthesis from a suitable optically pure precursor or resolution of the racemate using, for example, chiral high-performance liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art. Chiral compounds of the invention (and chiral precursors thereof) may be obtained in enantiomerically enriched form using chromatography, typically HPLC, on an asymmetric resin with a mobile phase consisting of a hydrocarbon, typically heptane or hexane, containing from 0% to 50% 2-propanol, typically from 2% to 20%, and from 0% to 5% of an alkylamine, typically 0.1% diethylamine. Concentration of the eluate affords the enriched mixture. Stereoisomeric conglomerates may be separated by conventional techniques known to those skilled in the art. See, e.g., Stereochemistry of Organic Compounds by E. L. Eliel and S. H. Wilen (Wiley, New York, 1994), the disclosure of which is incorporated herein by reference in its entirety. Suitable stereoselective techniques are well known to those of ordinary skill in the art.

Where a compound of the invention contains an alkenyl or alkenylene (alkylidene) group, geometric cis/trans (or Z/E) isomers are possible. Cis/trans isomers may be separated by conventional techniques well known to those skilled in the art, for example, chromatography and fractional crystallization. Salts of the present invention can be prepared according to methods known to those of skill in the art.

The compounds of the invention that are basic in nature are capable of forming a wide variety of salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the basic compounds of this invention can be prepared by treating the basic compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, isonicotinic acid, lactic acid, pantothenic acid, bitartric acid, ascorbic acid, 2,5-dihydroxybenzoic acid, gluconic acid, saccharic acid, formic acid, methanesulfonic acid, ethanesulfonic acid, benzenesulfonic acid, p-toluenesulfonic acid, and pamoic [i.e., 4,4′-methanediylbis(3-hydroxynaphthalene-2-carboxylic acid)] acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as ethanesulfonic acid, or the like.

Those compounds of the invention that are acidic in nature are capable of forming base salts with various pharmacologically acceptable cations. Examples of such salts include the alkali metal or alkaline earth metal salts, and particularly the sodium and potassium salts. These salts are all prepared by conventional techniques. The chemical bases which are used as reagents to prepare the pharmaceutically acceptable base salts of this invention are those which form non-toxic base salts with the acidic compounds of the invention. These salts may be prepared by any suitable method, for example, treatment of the free acid with an inorganic or organic base, such as an amine (primary, secondary or tertiary), an alkali metal hydroxide or alkaline earth metal hydroxide, or the like. These salts can also be prepared by treating the corresponding acidic compounds with an aqueous solution containing the desired pharmacologically acceptable cations, and then evaporating the resulting solution to dryness, for example under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are, for example, employed in order to ensure completeness of reaction and maximum yields of the desired final product.

Pharmaceutically acceptable salts of compounds of the invention (including compounds of the invention-a or I-b) may be prepared by, e.g., one or more of three methods:

(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionized to almost non-ionized.

Polymorphs can be prepared according to techniques well-known to those skilled in the art, for example, by crystallization.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture may have almost identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, New York, 1994).

The invention also includes isotopically labeled compounds of the invention wherein one or more atoms is replaced by an atom having the same atomic number, but an atomic mass or mass number different from the atomic mass or mass number usually found in nature. Isotopically labeled compounds of the invention (or pharmaceutically acceptable salts thereof or N-oxides thereof) can generally be prepared by conventional techniques known to those skilled in the art or by processes analogous to those described herein, using an appropriate isotopically labeled reagent in place of the non-labeled reagent otherwise employed.

Prodrugs in accordance with the invention can, for example, be produced by replacing appropriate functionalities present in the compounds of the invention with certain moieties known to those skilled in the art as ‘pro-moieties’ as described, for example, in Design of Prodrugs by H. Bundgaard (Elsevier, 1985).

The compounds of the invention should be assessed for their biopharmaceutical properties, such as solubility and solution stability (across pH), permeability, etc., in order to select the most appropriate dosage form and route of administration for treatment of the proposed indication.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention (or pharmaceutically acceptable salts thereof) and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention (including pharmaceutically acceptable salts thereof) may be administered orally. Oral administration may involve swallowing, so that the compound enters the gastrointestinal tract, and/or buccal, lingual, or sublingual administration by which the compound enters the bloodstream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast-dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropyl methyl cellulose) and typically comprise a carrier, for example, water, ethanol, polyethylene glycol, propylene glycol, methyl cellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described by Liang and Chen, Expert Opinion in Therapeutic Patents 2001, 11, 981-986.

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 weight % to 25 weight %, for example, from 5 weight % to 20 weight % of the dosage form. Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulfate. Lubricants generally comprise from 0.25 weight % to 10 weight %, for example, from 0.5 weight % to 3 weight % of the tablet.

Other possible ingredients include anti-oxidants, colorants, flavoring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt-congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of the invention, a film-forming polymer, a binder, a solvent, a humectant, a plasticizer, a stabilizer or emulsifier, a viscosity-modifying agent and a solvent. Some components of the formulation may perform more than one function.

The compound of the invention (or pharmaceutically acceptable salts thereof or N-oxides thereof) may be water-soluble or insoluble. A water-soluble compound typically comprises from 1 weight % to 80 weight %, more typically from 20 weight % to 50 weight %, of the solutes. Less soluble compounds may comprise a smaller proportion of the composition, typically up to 30 weight % of the solutes. Alternatively, the compound of the invention may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavorings and flavor enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al., Pharmaceutical Technology On-line, 25(2), 1-14 (2001). The use of chewing gum to achieve controlled release is described in WO 00/35298.

The compounds of the invention (including pharmaceutically acceptable salts thereof) may also be administered directly into the bloodstream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (for example to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of compounds of the invention (including pharmaceutically acceptable salts thereof) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(DL-lactic-coglycolic acid) (PLGA) microspheres.

The compounds of the invention (including pharmaceutically acceptable salts thereof) may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated. See e.g., Finnin and Morgan, J. Pharm. Sci. 1999, 88, 955-958.

Other means of topical administration include delivery by electroporation, iontophoresis, phonophoresis, sonophoresis and microneedle or needle-free (e.g., Powderject™, Bioject™ etc.) injection.

Formulations for topical administration may be formulated to be immediate and/or modified release. Modified release formulations include delayed-, sustained-, pulsed-, controlled-, targeted and programmed release.

The compounds of the invention (including pharmaceutically acceptable salts thereof) can also be administered intranasally or by inhalation, typically in the form of a dry powder (either alone; as a mixture, for example, in a dry blend with lactose; or as a mixed component particle, for example, mixed with phospholipids, such as phosphatidylcholine) from a dry powder inhaler, as an aerosol spray from a pressurized container, pump, spray, atomizer (for example an atomizer using electrohydrodynamics to produce a fine mist), or nebulizer, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules (made, for example, from gelatin or hydroxypropyl methyl cellulose), blisters and cartridges for use in an inhaler or insufflator may be formulated to contain a powder mix of the compound of the invention, a suitable powder base such as lactose or starch and a performance modifier such as L-leucine, mannitol, or magnesium stearate. The lactose may be anhydrous or in the form of the monohydrate. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 μg to 20 mg of the compound of the invention per actuation and the actuation volume may vary from 1 μL to 100 μL. A typical formulation may comprise a compound of the invention or a pharmaceutically acceptable salt thereof, propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 0.01 to 100 mg of the compound of the invention. The overall daily dose will typically be in the range 1 μg to 200 mg, which may be administered in a single dose or, more usually, as divided doses throughout the day.

The compounds of the invention (including pharmaceutically acceptable salts thereof) may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention (including pharmaceutically acceptable salts thereof) may also be administered directly to the eye or ear, typically in the form of drops of a micronized suspension or solution in isotonic, pH-adjusted, sterile saline. Other formulations suitable for ocular and aural administration include ointments, gels, biodegradable (e.g., absorbable gel sponges, collagen) and non-biodegradable (e.g., silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropyl methyl cellulose, hydroxyethyl cellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention (including pharmaceutically acceptable salts thereof) may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e., as a carrier, diluent, or solubilizer. Most commonly used for these purposes are alpha-, beta- and gamma-cyclodextrins, examples of which may be found in International Patent Applications Nos. WO 91/11172, WO 94/02518 and WO 98/55148.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of the invention, a prodrug thereof, or a salt of such compound or prodrug; and a second compound as described above. The kit comprises means for containing the separate compositions such as a container, a divided bottle or a divided foil packet. Typically the kit comprises directions for the administration of the separate components. The kit form is particularly advantageous when the separate components are for example administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. In some embodiments, the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen on which the tablets or capsules so specified should be ingested. Another example of such a memory aid is a calendar printed on the card, e.g., as follows “First Week, Monday, Tuesday, etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of the invention compound can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. For example, the dispenser is equipped with a memory aid, so as to further facilitate compliance with the regimen. An example of such a memory aid is a mechanical counter which indicates the number of daily doses that has been dispensed. Another example of such a memory aid is a battery-powered micro-chip memory coupled with a liquid crystal readout, or audible reminder signal which, for example, reads out the date that the last daily dose has been taken and/or reminds one when the next dose is to be taken.

The invention will be described in greater detail by way of specific examples. The following examples are offered for illustrative purposes, and are not intended to limit the invention in any manner. Those of skill in the art will readily recognize a variety of non-critical parameters that can be changed or modified to yield essentially the same results. Additional compounds within the scope of this invention may be prepared using the methods illustrated in these Examples, either alone or in combination with techniques generally known in the art. In the following Examples and Preparations, “DMSO” means dimethyl sulfoxide, “N” where referring to concentration means Normal, “M” means molar, “mL” means milliliter, “mmol” means millimoles, “μmol” means micromoles, “eq.” means equivalent, “° C.” means degrees Celsius, “MHz” means megahertz, “HPLC” means high-performance liquid chromatography.

### EXAMPLES

The following illustrate the synthesis of various compounds of the present invention. Additional compounds within the scope of this invention may be prepared using the methods illustrated in these Examples, either alone or in combination with techniques generally known in the art.

Experiments were generally carried out under inert atmosphere (nitrogen or argon), particularly in cases where oxygen- or moisture-sensitive reagents or intermediates were employed. Commercial solvents and reagents were generally used without further purification. Anhydrous solvents were employed where appropriate, generally AcroSeal® products from Acros Organics, Aldrich® Sure/Seal™ from Sigma-Aldrich, or DriSolv® products from EMD Chemicals. In other cases, commercial solvents were passed through columns packed with 4 Å molecular sieves, until the following QC standards for water were attained: a) <100 ppm for dichloromethane, toluene, N,N-dimethylformamide and tetrahydrofuran; b) <180 ppm for methanol, ethanol, 1,4-dioxane and diisopropylamine. For very sensitive reactions, solvents were further treated with metallic sodium, calcium hydride, or molecular sieves, and distilled just prior to use. Products were generally dried under vacuum before being carried on to further reactions or submitted for biological testing. Mass spectrometry data is reported from either liquid chromatography-mass spectrometry (LCMS), ultra-performance liquid chromatography-mass spectrometry (UPLC-MS), atmospheric pressure chemical ionization (APCI) or gas chromatography-mass spectrometry (GCMS) instrumentation. Chemical shifts for nuclear magnetic resonance (NMR) data are expressed in parts per million (ppm, δ) referenced to residual peaks from the deuterated solvents employed. In some examples, chiral separations were carried out to separate enantiomers of certain compounds of the invention (in some examples, the separated enantiomers are designated as ENT-1 and ENT-2, according to their order of elution). In some examples, the optical rotation of an enantiomer was measured using a polarimeter. According to its observed rotation data (or its specific rotation data), an enantiomer with a clockwise rotation was designated as the (+)-enantiomer and an enantiomer with a counter-clockwise rotation was designated as the (−)-enantiomer. Racemic compounds are indicated by the presence of (+/−) adjacent to the structure; in these cases, indicated stereochemistry represents the relative (rather than absolute) configuration of the compound's substituents.

Reactions proceeding through detectable intermediates were generally followed by LCMS, and allowed to proceed to full conversion prior to addition of subsequent reagents. For syntheses referencing procedures in other Examples or Methods, reaction conditions (reaction time and temperature) may vary. In general, reactions were followed by thin-layer chromatography or mass spectrometry, and subjected to work-up when appropriate. Purifications may vary between experiments: in general, solvents and the solvent ratios used for eluents/gradients were chosen to provide appropriate R_{f}s or retention times. All starting materials in these Preparations and Examples are either commercially available or can be prepared by methods known in the art or as described herein.

### Example 1

### 1,1,1,3,3,3-Hexafluoropropan-2-yl (3R)-3-{[(cyclopropylmethyl)sulfonyl]amino}-1-oxa-8-azaspiro[4.5]decane-8-carboxylate (1)

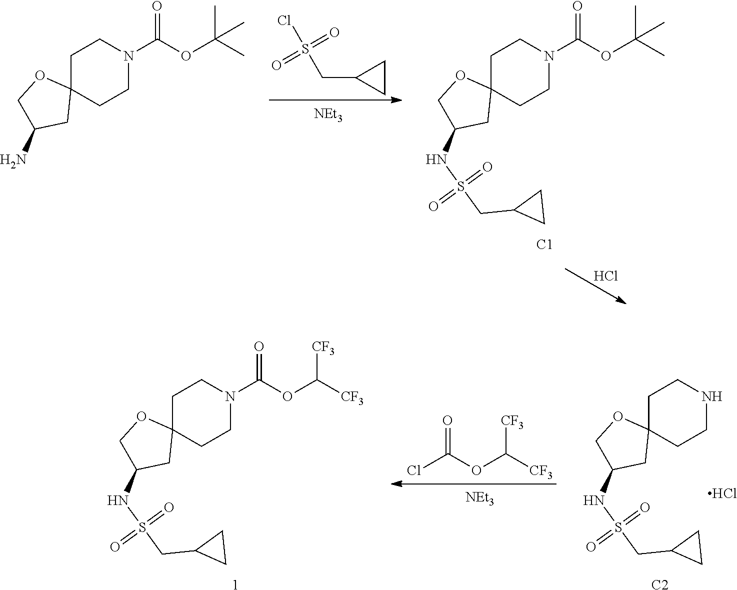

### Step 1. Synthesis of tert-butyl (3R)-3-{[(cyclopropylmethyl)sulfonyl]amino}-1-oxa-8-azaspiro[4.5]decane-8-carboxylate (C1)

A solution of tert-butyl (3R)-3-amino-1-oxa-8-azaspiro[4.5]decane-8-carboxylate (see C. R. Butler et al., U.S. Pat. Appl. Publ., 20170029390, Feb. 2, 2017) (3.00 g, 11.7 mmol) and triethylamine (4.08 mL, 29.3 mmol) in acetonitrile (9.0 mL) was cooled to 0° C. Cyclopropylmethanesulfonyl chloride (2.44 g, 15.8 mmol) was added drop-wise over 20 minutes, while the temperature of the reaction mixture was maintained below 7° C. It was then warmed to 25° C. and stirred at that temperature for 1.5 hours, at which time analysis by LCMS indicated 98% conversion to the product. The mixture was heated to 50° C. for 1 hour, whereupon water (9.0 mL) was added; the mixture was cooled to 0° C., seeding at 35° C., and was then maintained at 0° C. for 17 hours. More water (21.0 mL) was added drop-wise over 10 minutes, and the slurry was granulated for 30 minutes; filtration provided a solid, which was washed with water (2×6 mL) to afford the product as a pale yellow solid. Yield: 3.52 g, 98% pure via UPLC-MS analysis, 9.21 mmol, 79%. LCMS m/z 375.1 [M+H]⁺. ¹H NMR (400 MHz, DMSO-d₆) δ 7.34 (d, J=6.3 Hz, 1H), 4.00-3.88 (m, 2H), 3.51 (dd, J=8.0, 6.0 Hz, 1H), 3.46-3.36 (m, 2H), 3.29-3.14 (br m, 2H), 3.02-2.90 (m, 2H), 2.09 (dd, J=12.8, 8.0 Hz, 1H), 1.64 (dd, J=12.8, 6.8 Hz, 1H), 1.61-1.51 (m, 2H), 1.51-1.41 (m, 2H), 1.38 (s, 9H), 1.05-0.93 (m, 1H), 0.60-0.53 (m, 2H), 0.35-0.29 (m, 2H).

### Step 2. Synthesis of 1-cyclopropyl-N-[(3R)-1-oxa-8-azaspiro[4.5]dec-3-yl]methanesulfonamide, hydrochloride salt (C2)

A slurry of C1 (2.62 g, 99% mass purity, 6.93 mmol) in 2-propanol (23.6 mL) was heated to 50° C. The resulting solution was treated with a solution of hydrogen chloride in 2-propanol (5 M; 2.77 mL, 13.8 mmol), and stirring was continued at 50° C. for 16 hours. After the reaction mixture had been cooled to 25° C., solids were collected via filtration and washed with 2-propanol (2×5.2 mL), affording the product as a white solid. Yield: 1.88 g, 6.05 mmol, 87%. LCMS m/z 275.2 [M+H]⁺. ¹H NMR (400 MHz, DMSO-d₆) δ 9.14-8.83 (br s, 2H), 7.40 (d, J=6.3 Hz, 1H), 4.02-3.89 (m, 2H), 3.55 (dd, J=8.5, 6.0 Hz, 1H), 3.10-2.92 (m, 6H), 2.13 (dd, J=12.8, 8.0 Hz, 1H), 1.89-1.79 (m, 2H), 1.79-1.67 (m, 3H), 1.05-0.93 (m, 1H), 0.60-0.53 (m, 2H), 0.35-0.29 (m, 2H).

### Step 3. Synthesis of 1,1,1,3,3,3-hexafluoropropan-2-yl (3R)-3-{[(cyclopropylmethyl)sulfonyl]amino}-1-oxa-8-azaspiro[4.5]decane-8-carboxylate (1)

A mixture of C2 (1.50 g, 4.83 mmol) and triethylamine (3.10 mL, 22.2 mmol) in tert-butyl methyl ether (15.0 mL) was heated to an internal temperature of 40° C. 1,1,1,3,3,3-Hexafluoropropan-2-yl carbonochloridate (1.33 g, 5.77 mmol) was added drop-wise over 8 minutes, and the reaction mixture was stirred at 40° C. for 80 minutes, whereupon methanol (15.0 mL) was added, and further heat was applied to initiate distillation. Distillation was interrupted when the temperature of the mixture reached 65° C. (boiling point of methanol), and the reaction volume was approximately 9 mL. The mixture was then cooled to 45° C., and treated in a drop-wise manner with water (9.0 mL) over 5 minutes. Methanol was added in portions (3×3 mL) to provide a solution, which was cooled to 0° C. and held at that temperature overnight. The precipitated solid was collected via filtration and washed with water (20 mL) to provide the product as a white solid. Yield: 1.63 g, 98% pure by UPLC-MS, 3.41 mmol, 71%. LCMS m/z 469.2 [M+H]⁺. ¹H NMR (400 MHz, DMSO-d₆) δ 7.36 (d, J=6.3 Hz, 1H), 6.55 (septet, J_{HF}=6.5 Hz, 1H), 4.02-3.89 (m, 2H), 3.63-3.50 (m, 3H), 3.43-3.26 (m, 2H), 3.02-2.92 (m, 2H), 2.12 (dd, J=12.8, 8.0 Hz, 1H), 1.73-1.45 (m, 5H), 1.04-0.93 (m, 1H), 0.60-0.53 (m, 2H), 0.35-0.29 (m, 2H).

### Preparation of Form I (anhydrate crystal form) of 1,1,1,3,3,3-Hexafluoropropan-2-yl (3R)-3-{[(cyclopropylmethyl)sulfonyl]amino}-1-oxa-8-azaspiro[4.5]decane-8-carboxylate (1)

1,1,1,3,3,3-Hexafluoropropan-2-yl (3R)-3-{[(cyclopropylmethyl)sulfonyl]amino}-1-oxa-8-azaspiro[4.5]decane-8-carboxylate (1, 750 mg, 1.60 mmol) was dissolved in a minimum amount of methyl t-butyl ether (MTBE) at ˜50 C. Heptane was then added dropwise at 50° C. until the solution became cloudy. The resulting mixture was cooled slowly to room temperature and stirred at room temperature for an additional 24 hours. The resulting suspension was filtered by suction filtration to afford 700 mg (93%) of crystalline 1,1,1,3,3,3-hexafluoropropan-2-yl (3R)-3-{[(cyclopropylmethyl)sulfonyl]amino}-1-oxa-8-azaspiro[4.5]decane-8-carboxylate (1), designated as Form I, as a white solid.

### Acquisition of Differential Scanning Calorimetry Data for Form I of the Compound of Example 1

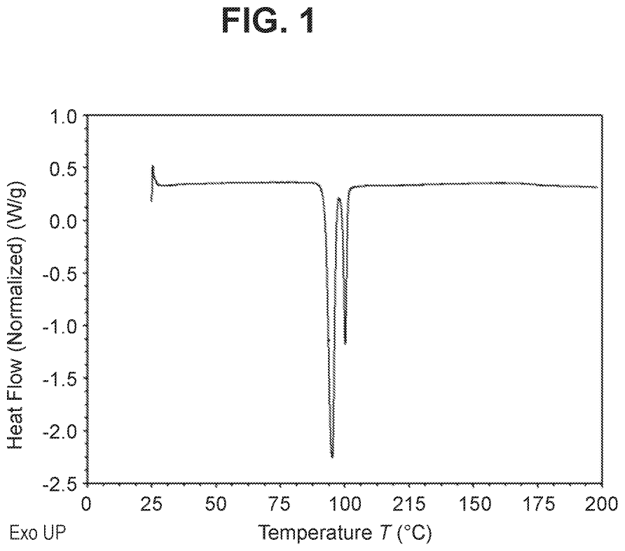

Differential scanning calorimetry (DSC) data (See FIG. 1) were collected using a Discovery DSC equipped with a refrigerated cooling accessory from TA instruments under the following parameters: All the experiments were performed in Tzero aluminum pans. The cell constant was determined using indium and temperature calibration was performed using indium and tin as standards. All the measurements were done under continuous dry nitrogen purge (50 mL/min). Approximately 2-5 mg of solid sample was weighed into a Tzero aluminum pan, sealed non-hermetically and heated from 25° C. to 200° C. at 10° C./min heating rates. The experimental data were analyzed using commercially available software (TA Universal Analysis 2000 software, TA Instruments).

As shown in FIG. 1, Differential Scanning calorimetry (DSC) data showed two melting endotherms with onset temperatures at about 91° C. and at about 97° C.

### Acquisition of Thermogravimetric Analysis Data for Form I of the Compound of Example 1

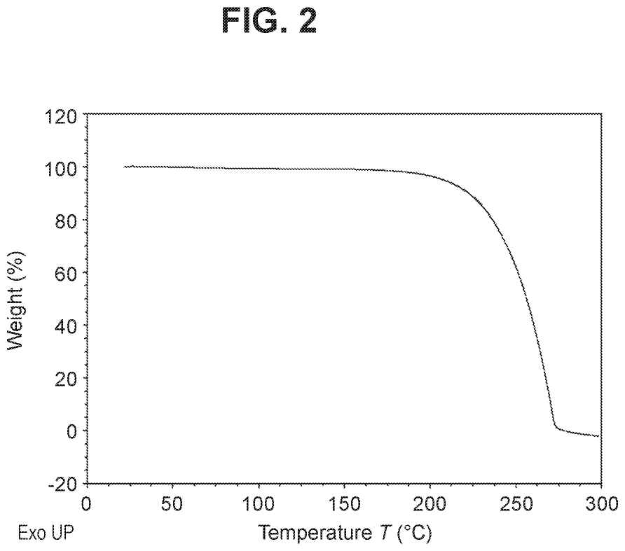

Thermogravimetric analysis data (see FIG. 2) was collected using a Discovery TGA instrument (TA instruments) under the following parameters: approximately 5 mg of samples were weighed into aluminum pans and heated from 25° C. to 300° C. at 10° C./minute heating rate under nitrogen purge (90 mL/min). As shown in FIG. 2, Thermogravimetric Analysis (TGA) showed no significant weight loss before the melting event, which confirms that the material was anhydrous (anhydrate), which is consisting with the DSC data.

### Acquisition of Dynamic Vapor Sorption Data for Form I of the Compound of Example 1

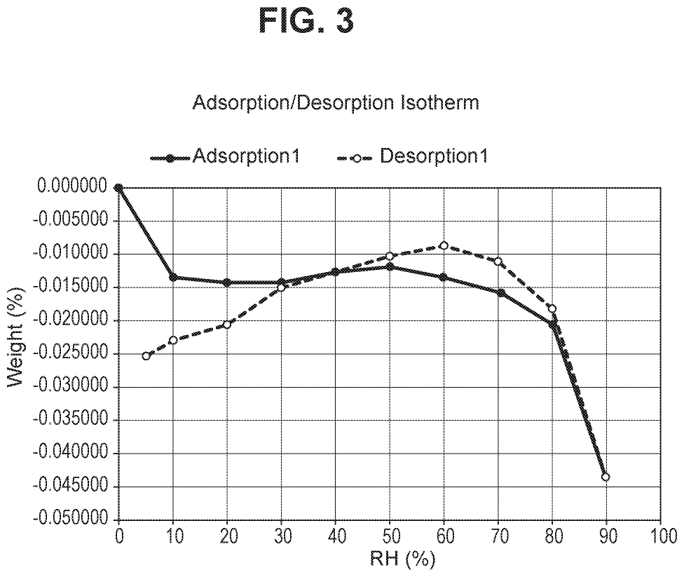

An automated vapor sorption analyzer (VTI SGA-CX; VTI scientific) was used to measure the hygroscopicity of the anhydrate of Form I of the invention. The microbalance was calibrated using a 100 mg standard weight. The relative humidity sensor was calibrated at 5.0, 11.3, 32.8, 52.8, 75.3, and 84.3% RH (25° C.) using saturated salt solutions, as well as 80% RH (25° C.) using polyvinylpyrrolidone. Approximately 8-10 mg of the powder sample was placed in the platinum sample pan and dried at 3% relative humidity (RH) at 25° C. with a heating rate of 1° C. per minute. The attainment of equilibrium was assumed when the weight change of the sample was <0.001 wt % in 5 min or by a maximum equilibration time of 120 minutes. The RH was then progressively increased to 90% in increments of 10% followed by a decreased to a final RH of 10% in 10% RH decrements. Again, the attainment of equilibrium was assumed when the weight change of the sample was <0.001 wt % in 5 min or by a maximum equilibration time of 120 minutes. The weight gain at the end of the sorption cycle (90% RH) was calculated on the basis of the dry weight. DVS data (see FIG. 3 and Table 2) reveal little weight gain (less than 0.1% at up to 90% RH, 25° C.) for Form I, indicating that this anhydrate (anhydrous) crystal form (Form I) is substantially non-hygroscopic.

### Acquisition of Powder X-Ray Diffraction (PXRD) Data for Form I of the Compound of Example 1

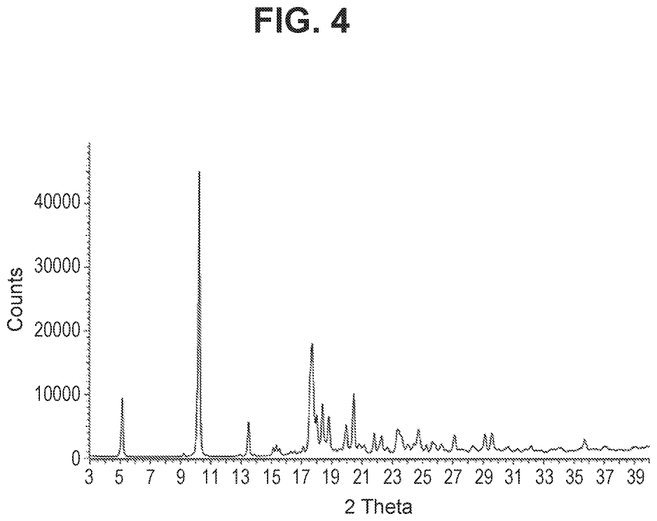

Powder X-ray diffraction (PXRD) data was acquired and collected on a sample of Form I of the compound of Example 1 using a Bruker AXS D8 Endeavor diffractometer equipped with a Cu radiation source (CuKα radiation, wavelength of 1.54056 Å). The divergence slit was set at 3 mm continuous illumination. Diffracted radiation was detected by a PSD-Lynx Eye detector, with the detector PSD opening set at 4.105 degrees. The X-ray tube voltage and amperage were set to 40 kV and 40 mA respectively. Data was collected in the Theta-Theta goniometer at the Cu wavelength from 3.0 to 40.0 degrees 2-Theta using a step size of 0.020 degrees and a step time of 0.5 second. Samples were prepared by placing them in a silicon low background sample holder and rotated during collection. Data were collected using Bruker DIFFRAC Plus software and analysis was performed by EVA diffract plus software. Generally a threshold value of 1° and a width value of 0.3° were used to make preliminary peak assignments. One diffraction pattern was consistently observed and is provided in FIG. 4. A list of diffraction peaks expressed in terms of the degree 2θ and relative intensities with a relative intensity of ≥4.0% is provided above in Table 1.

### Example 2

### 1,1,1,3,3,3-Hexafluoropropan-2-yl (3R)-3-[(cyclopropylsulfonyl)amino]-1-oxa-8-azaspiro[4.5]decane-8-carboxylate (2)

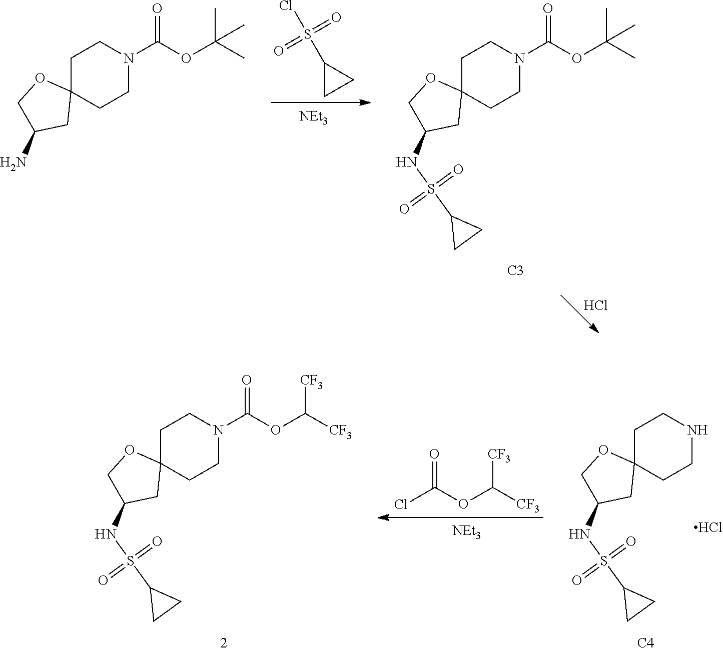

### Step 1. Synthesis of tert-butyl (3R)-3-[(cyclopropylsulfonyl)amino]-1-oxa-8-azaspiro[4.5]decane-8-carboxylate (C3)

A solution of tert-butyl (3R)-3-amino-1-oxa-8-azaspiro[4.5]decane-8-carboxylate (0.25 g, 0.98 mmol) and triethylamine (0.34 mL, 2.4 mmol) in methanol (1.0 mL) was cooled to 0° C. Cyclopropanesulfonyl chloride (0.12 mL, 98% mass purity, 1.2 mmol) was added drop-wise over 7 minutes, and the reaction mixture was allowed to stir for 5 hours, whereupon it was warmed to 25° C. After 12 hours, the slurry was heated to 50° C., providing a solution. Water (1.0 mL) was added slowly, over 1 minute; upon stirring for 5 minutes a 50° C., a thick slurry had formed. Water (1.5 mL) was added again, and the mixture was cooled to 25° C. and granulated for 30 minutes. The solid was collected via filtration and washed with water (2×2 mL, then 6 mL), affording the product as a solid. Yield: 0.29 g, 0.80 mmol, 82%. LCMS m/z 361.2 [M+H]⁺. ¹H NMR (400 MHz, DMSO-d₆) δ 7.34 (d, J=7.0 Hz, 1H), 4.03-3.91 (m, 2H), 3.57-3.50 (m, 1H), 3.46-3.37 (m, 2H), 3.29-3.15 (br m, 2H), 2.59-2.51 (m, 1H), 2.12 (dd, J=12.8, 8.3 Hz, 1H), 1.66 (dd, J=12.9, 6.4 Hz, 1H), 1.62-1.52 (m, 2H), 1.52-1.40 (m, 2H), 1.39 (s, 9H), 0.99-0.85 (m, 4H).

### Step 2. Synthesis of N-[(3R)-1-oxa-8-azaspiro[4.5]dec-3-yl]cyclopropanesulfonamide, hydrochloride salt (C4)

A mixture of C3 (3.67 g, 10.2 mmol) in 2-propanol (33.0 mL) was heated to 50° C., and the resulting solution was treated with a solution of hydrogen chloride in 2-propanol (5 M; 4.07 mL, 20.4 mmol). The reaction mixture was stirred, using an overhead stirrer, for 18 hours at 50° C., whereupon it was cooled to 25° C. The solids were collected via filtration and washed with 2-propanol (2×8 mL), providing the product as a white solid. Yield: 2.80 g, 9.43 mmol, 92%. LCMS m/z 261.1 [M+H]⁺. ¹H NMR (400 MHz, DMSO-d₆) δ 9.11-8.79 (br s, 2H), 7.40 (d, J=7.0 Hz, 1H), 4.05-3.92 (m, 2H), 3.58 (dd, J=8.4, 5.6 Hz, 1H), 3.11-2.91 (br m, 4H), 2.61-2.53 (m, 1H), 2.17 (dd, J=13.0, 8.0 Hz, 1H), 1.88-1.81 (m, 2H), 1.78-1.69 (m, 3H), 1.00-0.86 (m, 4H).

### Step 3. Synthesis of 1,1,1,3,3,3-hexafluoropropan-2-yl (3R)-3-[(cyclopropylsulfonyl)amino]-1-oxa-8-azaspiro[4.5]decane-8-carboxylate (2)

A solution of C4 (0.25 g, 0.84 mmol) and triethylamine (0.27 mL, 1.9 mmol) in methanol (1.0 mL) was cooled to 0° C. and treated in a drop-wise manner, over 6 minutes via syringe, with 1,1,1,3,3,3-hexafluoropropan-2-yl carbonochloridate (SynQuest Laboratories; 0.23 g, 1.0 mmol). After 50 minutes, the reaction mixture was heated to 50° C. and treated drop-wise with water (1.5 mL) over 3 minutes. After an additional 25 minutes, the mixture was cooled to 25° C.; at 43° C., it was seeded with 2 (Form A seed, see preparation below) (13 mg, 29 μmol). An even slurry was obtained upon stirring this mixture at 25° C. Water (1.0 mL) was added drop-wise, and the mixture was granulated for 30 minutes; filtration provided a filter cake, which was washed with water (2×0.75 mL) to afford the product as a white solid. Yield: 0.246 g, 0.541 mmol, 64%. LCMS m/z 472.2 [M+NH₄⁺]. ¹H NMR (400 MHz, DMSO-d₆) δ 7.36 (d, J=6.8 Hz, 1H), 6.55 (septet, J_{HF}=6.4 Hz, 1H), 4.05-3.92 (m, 2H), 3.65-3.51 (m, 3H), 3.44-3.25 (m, 2H), 2.61-2.52 (m, 1H), 2.15 (dd, J=12.9, 7.9 Hz, 1H), 1.75-1.45 (m, 5H), 1.00-0.85 (m, 4H).

### Preparation of Form A of the compound of Example 2 (1,1,1,3,3,3-hexafluoropropan-2-yl(3R)-3-[(cyclopropylsulfonyl)amino]-1-oxa-8-azaspiro[4.5]decane-8-carboxylate)

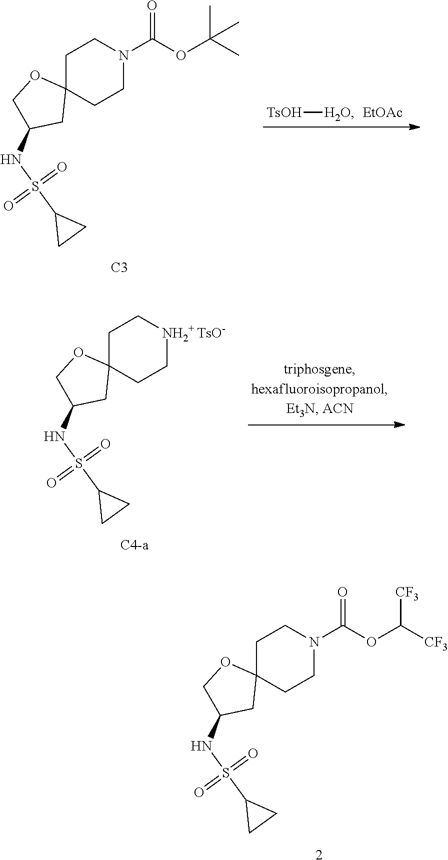

Tert-butyl (3R)-3-[(cyclopropylsulfonyl)amino]-1-oxa-8-azaspiro[4.5]decane-8-carboxylate (C3, 20.0 g, 55.5 mmol) was suspended in 270 mL of EtOAc and treated with 4-toluenesulfonic acid monohydrate (TSOH-H₂O; 15.8 g, 83.2 mmol). The resulting mixture was warmed to 50° C. and then subsequently cooled slowly to room temperature. The mixture was stirred at room temperature for 18 hours. Heptane (100 mL) was added to the resulting suspension and stirring was continued for an additional 30 minutes. The solids were collected by suction filtration, rinsed with 1:1 EtOAc/heptane, and dried under vacuum at 50° C. to afford 20.0 g (83%) of N-[(3R)-1-oxa-8-azaspiro[4.5]dec-3-yl]cyclopropanesulfonamide 4-toluenesulfonic acid salt (C4-a). ¹H NMR (400 MHz, DMSO-d₆) δ ppm 8.38 (br. s., 1H), 8.28 (br. s., 1H), 7.49 (d, J=8.0 Hz, 2H), 7.37 (d, J=7.0 Hz, 1H), 7.12 (d, J=8.0 Hz, 2H), 4.07-3.91 (m, 2H), 3.58 (dd, J=8.2, 5.9 Hz, 1H), 3.16-2.94 (m, 4H), 2.62-2.52 (m, 1H), 2.29 (s, 3H), 2.17 (dd, J=13.1, 8.0 Hz, 1H), 1.90-1.63 (m, 5H), 1.02-0.84 (m, 4H).

1,1,1,3,3,3-Hexafluoroisopropanol (6.75 mL, 64.2 mmol) and Et₃N (12.0 mL, 85.5 mmol) were added to a solution of triphosgene (6.48 g, 21.4 mmol) in acetonitrile (400 mL). The resulting mixture was stirred at room temperature for 3 hours. N-[(3R)-1-oxa-8-azaspiro[4.5]dec-3-yl]cyclopropanesulfonamide 4-toluenesulfonic acid salt (C4-a, 18.5 g, 42.8 mmol) and Et₃N (12.0 mL, 85.5 mmol) were added and the reaction was stirred at room temperature for 18 hours. The reaction mixture was concentrated under reduced pressure, diluted with water and extracted several times with EtOAc. The combined organic extracts were washed with brine, dried over MgSO₄, filtered, and concentrated under reduced pressure. The resulting residue was purified by flash chromatography on silica gel (25-50% EtOAc/heptanes) to afford 16 g of 1,1,1,3,3,3-hexafluoropropan-2-yl (R)-3-(cyclopropanesulfonamido)-1-oxa-8-azaspiro[4.5]decane-8-carboxylate as a viscous yellow oil, which slowly solidified upon standing. The product was dissolved in a minimum amount of MTBE at ˜50 C. Heptane was then added dropwise at 50 C until the solution became cloudy. The resulting mixture was cooled slowly to room temperature and stirred at room temperature for an additional 24 hours. The resulting suspension was filtered by suction filtration to afford 15.2 g (78%) of crystalline 1,1,1,3,3,3-hexafluoropropan-2-yl (3R)-3-[(cyclopropylsulfonyl)amino]-1-oxa-8-azaspiro[4.5]decane-8-carboxylate, designated as Form A, as a white solid. ¹H NMR (400 MHz, CHLOROFORM-d) δ ppm 5.76 (septet, J=6.2 Hz, 1H), 4.39 (d, J=8.2 Hz, 1H), 4.15 (m, 1H), 4.05 (dd, J=9.8, 5.9 Hz, 1H), 3.85 (m, 2H), 3.76 (dd, J=9.8, 4.7 Hz, 1H), 3.37 (m, 2H), 2.42 (m, 1H), 2.21 (dd, J=13.5, 7.6 Hz, 1H), 1.86-1.64 (m, 4H), 1.64-1.48 (m, 1H), 1.20 (m, 2H), 1.05 (m, 2H).

### Acquisition of Differential Scanning Calorimetry Data for Form a of the Compound of Example 2

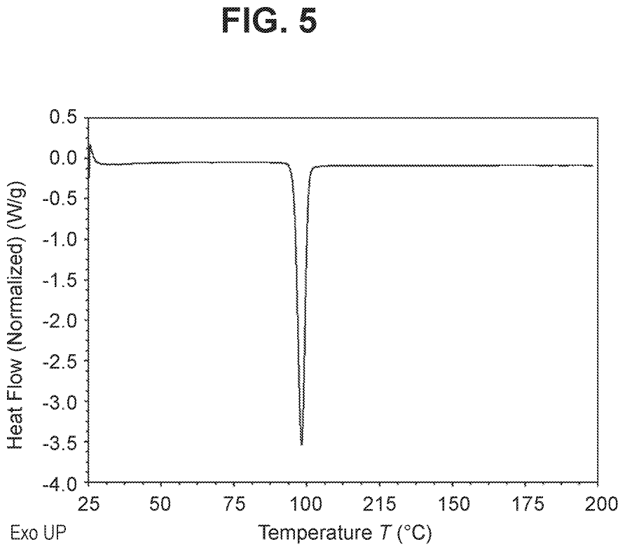

Differential scanning calorimetry data (See FIG. 5 and Table 4) were collected using a Discovery DSC equipped with a refrigerated cooling accessory from TA instruments under the following parameters: All the experiments were performed in Tzero aluminum pans. The cell constant was determined using indium and temperature calibration was performed using indium and tin as standards. All the measurements were done under continuous dry nitrogen purge (50 mL/min). Approximately 2-5 mg of solid sample was weighed into a Tzero aluminum pan, sealed non-hermetically and heated from 25° C. to 200° C. at 10° C./min heating rates. The experimental data were analyzed using commercially available software (TA Universal Analysis 2000 software, TA Instruments).

As shown in FIG. 5 and Table 4, Differential Scanning calorimetry (DSC) data showed one melting endotherm with onset temperature at about 96° C.

### Acquisition of Thermogravimetric Analysis Data for Form A of the Compound of Example 2

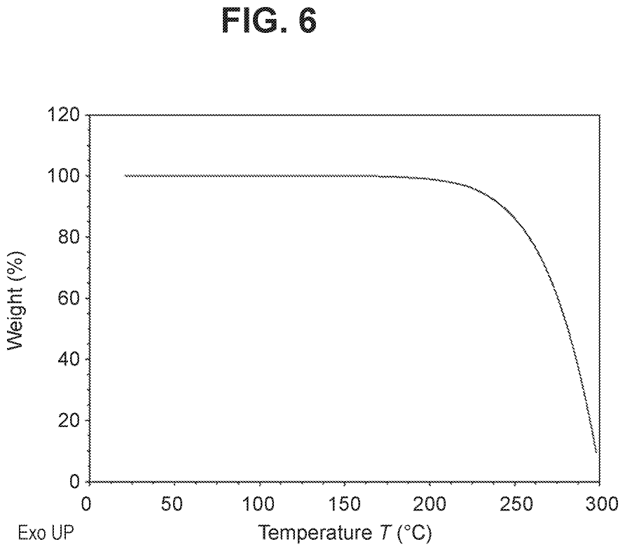

Thermogravimetric analysis data (see FIG. 6 and Table 4) was collected using a Discovery TGA instrument (TA instruments) under the following parameters: approximately 5 mg of samples were weighed into aluminum pans and heated from 25° C. to 300° C. at 10° C./minute heating rate under nitrogen purge (90 mL/min).

As shown in FIG. 6 and Table 4, Thermogravimetric Analysis (TGA) showed less than about 0.1% weight loss before the melting event, which confirms that the material was anhydrous (anhydrate), which is consisting with the DSC data (with no other event than the one melting endotherm event).

### Acquisition of Dynamic Vapor Sorption Data for Form A of the Compound of Example 2

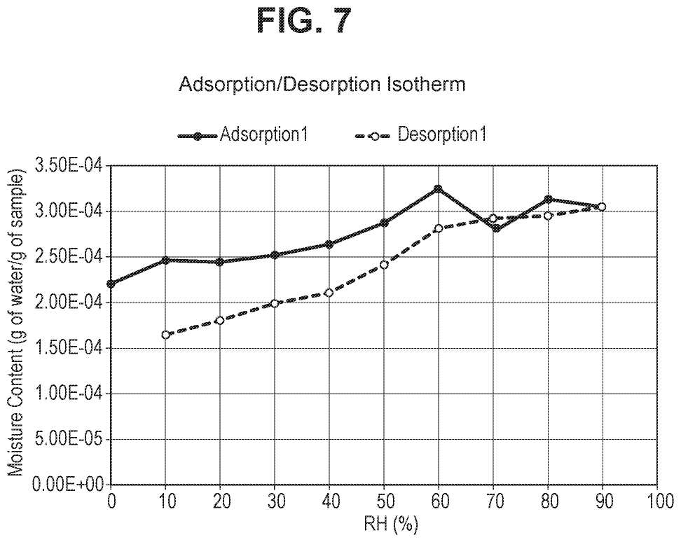

Moisture/Water sorption and desorption studies were conducted on automated vapor sorption analyzer (TA instruments Q5000 SA). The microbalance was calibrated using a 100 mg standard weight. The relative humidity sensor was calibrated at 5.0, 11.3, 32.8, 52.8, 75.3, and 84.3% RH (25° C.) using saturated salt solutions. Approximately 10-20 mg of the powder sample was placed in the platinum sample pan and dried at ≤3% relative humidity (RH) at 25° C. The attainment of equilibrium was assumed when the weight change of the sample was <0.001 wt % in 5 min or by a maximum equilibration time of 120 minutes. The RH was then progressively increased to 90% in increments of 10% followed by a decreased to a final RH of 10% in 10% RH decrements. Again, the attainment of equilibrium was assumed when the weight change of the sample was <0.001 wt % in 5 min or by a maximum equilibration time of 120 minutes. The weight gain at the end of the sorption cycle (90% RH) was calculated on the basis of the dry weight. DVS data (see FIG. 7 and Table 4) reveal little weight gain (less than about 0.1% at up to 90% RH, 25° C.) for Form A, indicating that Form A is largely non-hygroscopic.

### Acquisition of Powder X-Ray Diffraction (PXRD) Data for Form A of the Compound of Example 2

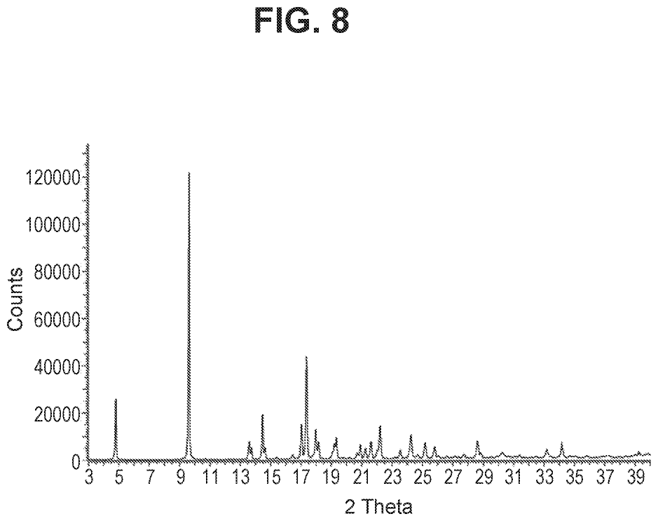

Powder X-ray diffraction (PXRD) data was acquired and collected on a sample of Form A of the compound of Example 2 using a Bruker AXS D8 Endeavor diffractometer equipped with a Cu radiation source (CuKα radiation, wavelength of 1.54056 Å). The divergence slit was set at 3 mm continuous illumination. Diffracted radiation was detected by a PSD-Lynx Eye detector, with the detector PSD opening set at 4.105 degrees. The X-ray tube voltage and amperage were set to 40 kV and 40 mA respectively. Data was collected in the Theta-Theta goniometer at the Cu wavelength from 3.0 to 40.0 degrees 2-Theta using a step size of 0.020 degrees and a step time of 0.5 second. Samples were prepared by placing them in a silicon low background sample holder and rotated during collection. Data were collected using Bruker DIFFRAC Plus software and analysis was performed by EVA diffract plus software. Generally a threshold value of 1° and a width value of 0.3° were used to make preliminary peak assignments. One diffraction pattern was consistently observed and is provided in FIG. 8. A list of diffraction peaks expressed in terms of the degree 2θ and relative intensities with a relative intensity of 4.0% is provided above in Table 3.

### Examples 3, 4, and 5

### 1,1,1,3,3,3-Hexafluoropropan-2-yl 3-{[(cyclopropylmethyl)sulfonyl]amino}-1-oxa-9-azaspiro[5.5]undecane-9-carboxylate (3), 1,1,1,3,3,3-Hexafluoropropan-2-yl 3-{[(cyclopropylmethyl)sulfonyl]amino}-1-oxa-9-azaspiro[5.5]undecane-9-carboxylate, ENT-1 (4), and 1,1,1,3,3,3-Hexafluoropropan-2-yl 3-{[(cyclopropylmethyl)sulfonyl]amino}-1-oxa-9-azaspiro[5.5]undecane-9-carboxylate, ENT-2 (5)

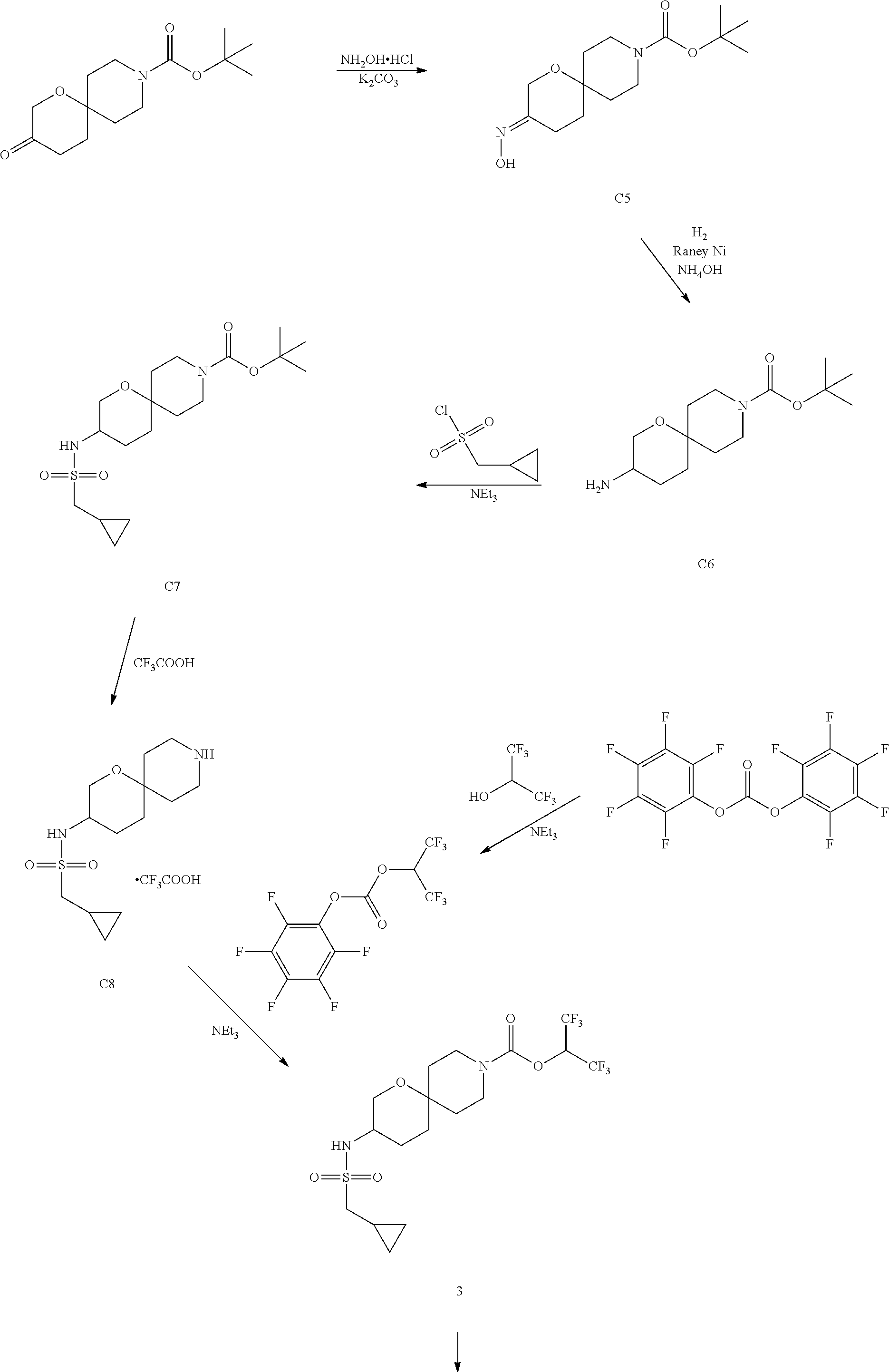

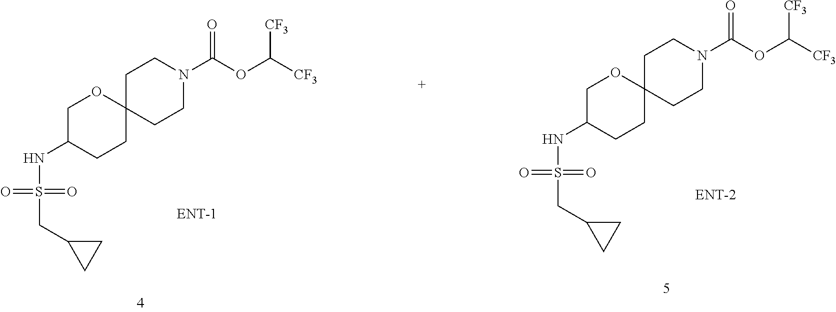 [SMILES: O=C(OC(C(F)(F)F)C(F)(F)F)N1CCC2(CCC(NS(=O)(=O)CC3CC3)CO2)CC1 ; O=C(OC(C(F)(F)F)C(F)(F)F)N1CCC2(CCC(NS(=O)(=O)CC3CC3)CO2)CC1]

### Step 1. Synthesis of tert-butyl 3-(hydroxyimino)-1-oxa-9-azaspiro[5.5]undecane-9-carboxylate (C5)

Hydroxylamine hydrochloride (1.74 g, 25.0 mmol) and potassium carbonate (6.93 g, 50.1 mmol) were added to a suspension of tert-butyl 3-oxo-1-oxa-9-azaspiro[5.5]undecane-9-carboxylate (4.5 g, 16.7 mmol) in ethanol (80 mL), and the reaction mixture was stirred at room temperature (25° C.) for 18 hours. Ethyl acetate (80 mL) was added, the resulting mixture was filtered, and the filtrate was concentrated in vacuo to afford the product as a colorless oil (6 g), which was used directly in the following step.

### Step 2. Synthesis of tert-butyl 3-amino-1-oxa-9-azaspiro[5.5]undecane-9-carboxylate (C6)

Raney nickel (1.57 g) was added to a solution of C5 (from the previous step; 6 g, mmol) in a mixture of methanol (80 mL) and ammonium hydroxide solution (80 mL). The reaction mixture was stirred under hydrogen (20 psi) for 7 hours at room temperature (25° C.), and then left standing at room temperature for 12 hours, whereupon it was stirred under hydrogen (20 psi) for 5 hours, and allowed to stand for an additional 18 hours. The mixture was filtered through diatomaceous earth, the filter pad was washed with methanol (150 mL), and the combined filtrates were concentrated in vacuo. Dilution of the residue with dichloromethane (60 mL) was followed by filtration; removal of solvent from this filtrate via concentration under reduced pressure afforded the product as a pale yellow oil (4.85 g). This material was used directly in the following step. ¹H NMR (400 MHz, CDCl₃), characteristic peaks: δ 3.84-3.40 (m, 4H), [3.27-2.96 (m) and 2.88-2.79 (m), total 3H], 1.46 (s, 9H).

### Step 3. Synthesis of tert-butyl 3-{[(cyclopropylmethyl)sulfonyl]amino}-1-oxa-9-azaspiro[5.5]undecane-9-carboxylate (C7)

To a 0° C. solution of C6 [from the previous step, 4.85 g, ≤16.7 mmol; combined with 850 mg, ≤2.49 mmol, synthesized in a similar manner] and triethylamine (10.7 mL, 76.8 mmol) in dichloromethane (80 mL) was added cyclopropylmethanesulfonyl chloride (4.45 g, 28.8 mmol). The reaction mixture was stirred at room temperature (25° C.) for 16 hours, whereupon it was concentrated in vacuo. The residue was purified using chromatography on silica gel (Gradient: 0% to 30% ethyl acetate in petroleum ether) to afford the product as a yellow solid. Yield: 3.5 g, 9.00 mmol, 47% over three steps. LCMS m/z 411.2 [M+Na⁺]. ¹H NMR (400 MHz, CDCl₃) δ 4.66-4.51 (br m, 1H), 3.87-3.64 (m, 3H), 3.54-3.44 (m, 2H), 3.18-3.01 (m, 2H), 2.95 (d, J=7.3 Hz, 2H), 2.00-1.61 (m, 5H), 1.54-1.35 (m, 3H), 1.46 (s, 9H), 1.22-1.09 (m, 1H), 0.75-0.68 (m, 2H), 0.44-0.37 (m, 2H).

### Step 4. Synthesis of 1-cyclopropyl-N-(1-oxa-9-azaspiro[5.5]undec-3-yl)methanesulfonamide, trifluoroacetate salt (C8)

Trifluoroacetic acid (6 mL) was added to a 0° C. solution of C7 (1.66 g, 4.27 mmol) in dichloromethane (30 mL). After the reaction mixture had been stirred at room temperature (25° C.) for 1 hour, it was concentrated in vacuo, affording the product as a yellow oil (2.7 g). This material was advanced directly into the following step. LCMS m/z 289.2 [M+H]⁺. ¹H NMR (400 MHz, CDCl₃), characteristic peaks: δ 8.46-8.19 (br s, 2H), 4.88-4.66 (br m, 1H), 3.88-3.77 (m, 1H), 2.97 (d, J=6.8 Hz, 2H), 2.21-2.04 (m, 2H), 2.02-1.91 (m, 1H), 1.84-1.68 (m, 3H), 1.64-1.52 (m, 1H), 1.21-1.09 (m, 1H), 0.77-0.69 (m, 2H), 0.45-0.37 (m, 2H).

### Step 5. Synthesis of 1,1,1,3,3,3-hexafluoropropan-2-yl 3-{[(cyclopropylmethyl)sulfonyl]amino}-1-oxa-9-azaspiro[5.5]undecane-9-carboxylate (3)

Bis(pentafluorophenyl) carbonate (3.43 g, 8.70 mmol) was added to a 0° C. solution of 1,1,1,3,3,3-hexafluoropropan-2-ol (1.44 g, 8.57 mmol) in acetonitrile (40 mL). Triethylamine (5.95 mL, 42.7 mmol) was added, and the reaction mixture was stirred at 0° C. for 30 minutes, then at 25° C. for 2 hours, providing Solution A.

Meanwhile, triethylamine (2.98 mL, 21.4 mmol) was added to a 0° C. solution of C8 (from the previous step; 2.7 g, ≤4.27 mmol) in acetonitrile (60 mL). After this reaction mixture had been stirred for 10 minutes at 0° C., Solution A (containing 1,1,1,3,3,3-hexafluoropropan-2-yl pentafluorophenyl carbonate) was added, and the reaction mixture was then allowed to warm and stir at 25° C. for 17 hours. It was combined with similar reactions carried out using C8 (380 mg, ≤0.695 mmol and 2.00 g, ≤3.35 mmol), and concentrated in vacuo. After the residue had been diluted with ethyl acetate (120 mL), it was washed with saturated aqueous sodium chloride solution (3×70 mL), dried over sodium sulfate, filtered, and concentrated under reduced pressure. Silica gel chromatography (Gradient: 0% to 30% ethyl acetate in petroleum ether) provided the product as a white solid. Combined yield: 3.21 g, 6.65 mmol, 80% over 2 steps. LCMS m/z 505.1 [M+Na⁺]. ¹H NMR (400 MHz, CDCl₃) δ 5.76 (septet, J_{HF}=6.3 Hz, 1H), 4.66-4.52 (m, 1H), 3.96-3.78 (m, 3H), 3.56-3.45 (m, 2H), 3.33-3.13 (m, 2H), 2.96 (d, J=6.8 Hz, 2H), 2.02-1.87 (m, 3H), 1.81-1.64 (m, 2H), 1.57-1.37 (m, 3H), 1.22-1.10 (m, 1H), 0.76-0.69 (m, 2H), 0.45-0.37 (m, 2H).

### Step 6. Isolation of 1,1,1,3,3,3-hexafluoropropan-2-yl 3-{[(cyclopropylmethyl)sulfonyl]amino}-1-oxa-9-azaspiro[5.5]undecane-9-carboxylate, ENT-1 (4) and 1,1,1,3,3,3-hexafluoropropan-2-yl 3-{[(cyclopropylmethyl)sulfonyl]amino}-1-oxa-9-azaspiro[5.5]undecane-9-carboxylate, ENT-2 (5)

Separation of 3 (3.2 g, 6.6 mmol) into its component enantiomers was carried out using supercritical fluid chromatography (Column: Phenomenex Lux Cellulose-2, 5 μm; Mobile phase: 92.5:7.5 carbon dioxide/methanol; Back pressure: 120 bar). The first-eluting enantiomer was designated as 4, and the second-eluting enantiomer as 5; both were isolated as solids.

4—Yield: 1.26 g, 2.61 mmol, 39% for the separation. ¹H NMR (400 MHz, CDCl₃) δ 5.75 (septet, J_{HF}=6.2 Hz, 1H), 4.70-4.55 (m, 1H), 3.96-3.78 (m, 3H), 3.56-3.44 (m, 2H), 3.33-3.13 (m, 2H), 2.96 (d, J=7.0 Hz, 2H), 2.02-1.87 (m, 3H), 1.80-1.64 (m, 2H), 1.57-1.37 (m, 3H), 1.22-1.10 (m, 1H), 0.76-0.68 (m, 2H), 0.44-0.37 (m, 2H). Retention time: 3.47 minutes (Analytical conditions. Column: Phenomenex Lux Cellulose-2, 250×4.6 mm; 5 μm; Mobile phase A: carbon dioxide; Mobile phase B: methanol; Gradient: 5% B for 1 minute, then 5% to 60% B over 8 minutes; Flow rate: 3.0 mL/minute; Back pressure: 120 bar).

5—Yield: 1.41 g, 2.92 mmol, 44% for the separation. ¹H NMR (400 MHz, CDCl₃) δ 5.75 (septet, J_{HF}=6.2 Hz, 1H), 4.71-4.56 (m, 1H), 3.96-3.78 (m, 3H), 3.56-3.44 (m, 2H), 3.33-3.13 (m, 2H), 2.96 (d, J=7.0 Hz, 2H), 2.02-1.87 (m, 3H), 1.80-1.64 (m, 2H), 1.57-1.37 (m, 3H), 1.22-1.10 (m, 1H), 0.76-0.69 (m, 2H), 0.44-0.37 (m, 2H). Retention time: 3.72 minutes (Analytical conditions identical to those used for 4).

### Example AA: MAGL and FAAH Enzymatic Assays

Assessment of MAGL inhibition utilizes human recombinant Monoacylglycerol Lipase and the fluorogenic substrate 7-hydroxycoumarinyl arachidonate (7-HCA, Biomol ST-502). 400 nL of a test compound at decreasing concentration (ranging from 150 μM down to 1.5 nM) was spotted into a 384-well back plate (PerkinElmer, 6007279) using a Labcyte Echo, followed by addition of 10 μL of MAGL enzyme in assay buffer (50 mM HEPES, pH 7.4, 100 mM NaCl, 5 mM MgCl₂, 0.1% Triton X-100 and 25% glycerin). An equal volume of 7-HCA in assay buffer with 10% DMSO was added either immediately (T=0 min) or after a 30 minute incubation (T=30 min) to initiate the reaction. The final concentration of MAGL enzyme was 88 μM and 7-HCA substrate was 5 μM. After these dilutions, the final concentration of the test compound ranged from 3 μM to 0.03 nM. The reaction was allowed to progress for 60 minutes, after which the plate was read at an Ex/Em of 340/465. Percent inhibitions were calculated based on control wells containing no compound (0% inhibition) and a control compound (e.g., a MAGL inhibitor whose activity is known or was previously reported in the literature, such as one with about 100% inhibition). IC₅₀ values were generated based on a four parameter fit model using ABASE software from IDBS. See e.g., Wang, Y. et al., “A Fluorescence-Based Assay for Monoacylglycerol Lipase Compatible with Inhibitor Screening,” Assay and Drug Development Technologies, 2008, Vol. 6 (3) pp 387-393 (reporting an assay for measuring MAGL activity).

To measure MAGL inactivation, the same protocol for the (T=0 min) MAGL inhibition IC₅₀ assay was performed with data collected every minute to acquire enzyme progress curves at decreasing concentrations of compound. K_{obs} values were calculated from this data and k_{inact}/Kₗ ratios were determined from a plot of K_{obs} values vs. compound concentrations.

Assessment of FAAH inhibition utilizes human recombinant FAAH and the fluorescent substrate, Arachidonoyl-AMC. 400 nL of a test compound at decreasing concentrations was spotted into a 384-well back plate (PerkinElmer, 6007279) using a Labcyte Echo, followed by addition of 10 μl of FAAH enzyme (Cayman 10010183) in assay buffer (50 mM Tris, pH 9.0, 1 mM EDTA, i.e. ethylenediaminetetraacetic acid). After a 30 minute incubation at room temperature, 10 μL of Arachidonyl-AMCA was added in assay buffer with 16% DMSO. Final concentration of FAAH enzyme was 0.0125 Units and AAMCA substrate was used at the Kₘ of 5 μM. After these dilutions, the final concentration of the test compound ranged from 3 μM to 0.03 nM. The reaction was allowed to progress for 60 minutes, after which the plate was read on a Molecular Devices FlexStation reader at an Ex/Em of 355/460. Percent inhibitions were calculated based on controls wells containing either no compound (0% inhibition) or a control compound (e.g., an FAAH inhibitor whose activity is known or was previously reported in the literature, such as one with about 100% inhibition). IC₅₀ values were generated based on a four parameter fit model using ABASE software from IDBS.

TABLE AA-1
Biological Data (MAGL IC₅₀, FAAH IC₅₀, and MAGL k_{inact}/K_{I}) for Examples 1-5.

| | MAGL | | MAGL | | FAAH | | MAGL | | |
|---|---|---|---|---|---|---|---|---|---|
| | (T = 0 min) | | (T = 30 min) | | (T = 30 min) | | k_{inact}/K_{I} | | |
| Example | IC₅₀ | | IC₅₀ | | IC₅₀ | | (1/s per | | |
| Number | (nM)^{a} | | (nM)^{a} | | (μM)^{a} | | M)^{a} | | Compound Name |
| 1 | 292 | (10) | 27.6 | (10) | >30 | (10) | 2990 | (12) | 1,1,1,3,3,3- |
| | | | | | | | | | hexafluoropropan-2-yl (3R)-3- |
| | | | | | | | | | {[(cyclopropylmethyl)sulfonyl]amino}- |
| | | | | | | | | | 1-oxa-8-azaspiro[4.5]decane-8- |
| | | | | | | | | | carboxylate |
| 2 | 369 | (10) | 34.7 | (10) | >30 | (8) | 2050 | (13) | 1,1,1,3,3,3- |
| | | | | | | | | | hexafluoropropan-2-yl (3R)-3- |
| | | | | | | | | | [(cyclopropylsulfonyl)amino]- |
| | | | | | | | | | 1-oxa-8-azaspiro[4.5]decane- |
| | | | | | | | | | 8-carboxylate |
| 3 | 298 | (6) | 28.5 | (6) | >30 | (8) | 3160 | (8) | 1,1,1,3,3,3- |
| | | | | | | | | | hexafluoropropan-2-yl 3- |
| | | | | | | | | | {[(cyclopropylmethyl)sulfonyl]amino}- |
| | | | | | | | | | 1-oxa-9-azaspiro[5.5]undecane-9- |
| | | | | | | | | | carboxylate |
| 4 | 162 | (2) | 20 | (2) | >30 | (4) | 3110 | (4) | 1,1,1,3,3,3- |
| | | | | | | | | | hexafluoropropan-2-yl 3- |
| | | | | | | | | | {[(cyclopropylmethyl)sulfonyl]amino}- |
| | | | | | | | | | 1-oxa-9-azaspiro[5.5]undecane-9- |
| | | | | | | | | | carboxylate, ENT-1 |
| 5 | 103 | (2) | 10.1 | (2) | >30 | (4) | 6930 | (4) | 1,1,1,3,3,3- |
| | | | | | | | | | hexafluoropropan-2-yl 3- |
| | | | | | | | | | {[(cyclopropylmethyl)sulfonyl]amino}- |
| | | | | | | | | | 1-oxa-9-azaspiro[5.5]undecane-9- |
| | | | | | | | | | carboxylate, ENT-2 |

^{a}Reported IC₅₀ values or k_{inact}/K_{I} values represent the geometric mean; the number of determinations is given in parentheses

### Example BB. Dog Pharmacokinetic Studies

The pharmacokinetic studies in dog were conducted at Pfizer Global Research and Development (Groton, Conn.). All in vivo studies were conducted in accordance with regulations and established guidelines using protocols reviewed and approved by the Pfizer Worldwide Research and Development (WRD, or other) Institutional Animal Care and Use Committee.

### Dog Intravenous Pharmacokinetic Study

Male beagle dogs were fasted overnight with water provided ad libitium. The dogs were fed at approximately 2-4 h post-dose. The dogs (n=2) were administered an IV bolus via the cephalic vein. Compounds of Examples 1-3 were tested along with the following four comparative compounds.

TABLE BB-1
Comparative Compounds

| Comparative- | Comparative- | Comparative- | |
|---|---|---|---|
| Compound | Compound | Compound | |
| Number | Name | Structure | Source |
| Comparative- Compound 1 | (2R)-1,1,1-trifluoro-3- hydroxypropan-2-yl (3R)-3- {[(cyclopropylmethyl)sul- fonyl](methyl)amino}-1- oxa-8- azaspiro[4.5]decane-8- carboxylate | 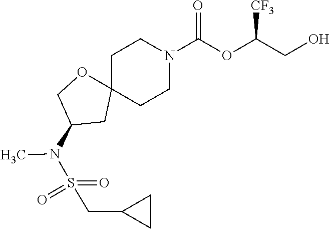 [SMILES: CN([C@H]1COC2(CCN(C(=O)O[C@H](CO)C(F)(F)F)CC2)C1)S(=O)(=O)CC1CC1] | Example 106 of U. S. Pat. No. 9,845,301 |
| Comparative- Compound 2 | (2R)-1,1,1-trifluoro-3- hydroxypropan-2-yl (3R)-3- [(cyclopropylsulfonyl)- (methyl)amino]-1-oxa-8- azaspiro[4.5]decane-8- carboxylate | 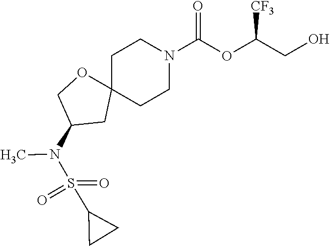 [SMILES: CN([C@H]1COC2(CCN(C(=O)O[C@H](CO)C(F)(F)F)CC2)C1)S(=O)(=O)C1CC1] | Example 93 of U. S. Pat. No. 9,845,301 |
| Comparative- Compound 3 | (2R)-1,1,1-trifluoro-3- hydroxypropan-2-yl (3R)-3- [methyl(phenylsulfonyl)- amino]-1-oxa-8- azaspiro[4.5]decane-8- carboxylate | 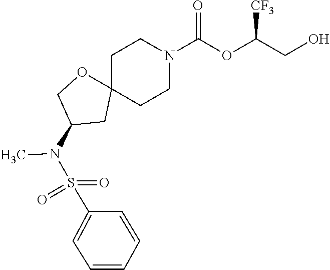 [SMILES: CN([C@H]1COC2(CCN(C(=O)O[C@H](CO)C(F)(F)F)CC2)C1)S(=O)(=O)c1ccccc1] | Example 15 of U. S. Pat. No. 9,845,301 |
| Comparative- Compound 4 | 1,1,1,3,3,3- hexafluoropropan-2-yl (3R)-3- [(phenylsulfonyl)amino]- 1-oxa-8- azaspiro[4.5]decane-8- carboxylate | 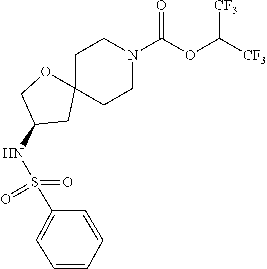 [SMILES: O=C(OC(C(F)(F)F)C(F)(F)F)N1CCC2(CC1)C[C@@H](NS(=O)(=O)c1ccccc1)CO2] | Example 32 of PCT/IB2018/050128 |

All test compounds were administered an IV dose of 1 mg/kg of the specified compound which was delivered in a dose volume of 0.5 mL/kg. The dose solution was formulated in 10% PEG400/90% of 23% (w/v) HPBCD in de-ionized water for the following test compounds: Example 1, Example 2, and Example 3; in 20% SBECD (sulfobutyl either β-cyclodextrin) for Comparative-Compound 3; in 23% (w/v) HPBCD (hydroxypropyl β-cyclodextrin) in water for Comparative Compound 2; in 12.5.% SBECD in water with 0.25% 0.1N HCl to pH 5 for Comparative Compound 1. Serial blood samples were collected from each dog via the jugular vein prior to dose administration and at the following time points post-dose: 0.083, 0.25, 0.5, 1, 2, 4, 7, and 24 h for all compounds with an additional time point of 0.016 h collected for Comparative Compound 1 only. Blood samples were collected into tubes containing EDTA and placed on wet ice. Following centrifugation to afford plasma, the samples were transferred to polypropylene tubes and stored frozen at −20° C. to −80° C. until analysis.

### Analysis of Compound in Plasma and Pharmacokinetic Parameters

All plasma samples were quantified via liquid chromatography/tandem mass spectrometry (LC/MS-MS) using multiple reaction monitoring with non-validated methods. The plasma standard curves were generated with species specific plasma. Pharmacokinetic parameters were determined by non-compartmental analysis using Watson LIMS software version 7.4 (Thermo Fisher Scientific, Waltham, Mass.). The area under the concentration versus time curve (AUC) was calculated from the first time point to the last time point with measurable drug concentration using a linear trapezoidal rule. The area under the concentration versus time curve from the first time point to time infinity (AUC_{(0-inf)}) was calculated by extrapolation from the time point where the last measurable concentration occurred to time infinity by dividing the observed concentration at the last time point by the elimination rate constant determined using linear regression of plasma concentration versus time data. The plasma clearance (CLₚ) was calculated by the equation CLₚ=dose/AUC_{(0-inf)}. The elimination half-life (t½) was calculated using the following equation t_{1/2}=ln 2/k; k is equal to the rate constant of elimination calculated by the least squares of the log transformed data. Data obtained are shown in Table 5 below.

### Example CC. In Vitro Intrinsic Clearance Studies in Human Liver Microsomes (HLM)

Test Compounds (Example 1-3 and Comparative-Compounds 1-4) were prepared as solutions in DMSO. The final concentration of DMSO in the incubation was <0.1% (v/v). The in vitro half-life (t½) of each compound was determined in incubations containing substrate (1 μM), human liver microsomes (P450 concentration, 0.25 μM) in 0.1 M potassium phosphate buffer (pH 7.4) with 3.3 mM magnesium chloride, and 1.3 mM NADPH (nicotinamide adenine dinucleotide phosphate) at 37° C. (all final concentrations) in a 384-well plate. The reaction mixture was pre-warmed at 37° C. for 10 min prior to initiation of the incubation. The incubation was quenched with acetonitrile containing internal standards at the following time points: 1, 4, 7, 12, 20, 25, 45, and 60 min (individual incubation plates per time point) with a subsequent addition of water. The samples were vortexed followed by centrifugation and the supernatant was analyzed by LC/MS-MS using multiple reaction monitoring. For control experiments, NADPH was omitted from the incubations.

The microsomal t½ (min) was obtained from a log-linear plot of the substrate depletion versus incubation time and was scaled to hepatic intrinsic clearance (CLint,app (μL/min/mg protein) using the following equation:

$$C {L}_{int , app} = ln 2 \cdot \frac{1}{{t}_{1 / 2} ( min )} \cdot \frac{mL incubation}{mg of microsomal protein}$$

Data obtained are shown in Table 5 below.

### Example DD. In Vitro Intrinsic Clearance Studies in Human Hepatocytes

Test Compounds (Example 1-3 and Comparative-Compounds 1-4) were prepared as solutions in DMSO at 3 mM which were then diluted in media containing WEM. The final concentration of DMSO in the incubation was 0.03% (v/v). The in vitro half-life (t½) of each compound was determined in incubations containing substrate (1 μM), human hepatocytes (0.5 million viable cells per mL final concentration) in media containing WEM, 50 mM HEPES buffer (pH 7.4), and 26 mM sodium bicarbonate at 37° C. (all final concentrations) in a 96-well plate. The reaction mixture was pre-warmed at 37° C. for 30 min prior to the initiation of the incubation. The incubation was quenched with acetonitrile containing internal standards at the following time points: 0, 5, 15, 30, 60, 120, and 240 min (individual incubation plates per time point). The samples were vortexed followed by centrifugation. An aliquot of supernatant was transferred to a clean 96-well plate and analyzed by LC/MS-MS using multiple reaction monitoring. For control experiments, hepatocytes were omitted from the incubations.

The hepatocyte t½ (min) was obtained from a log-linear plot of the substrate depletion versus incubation time and was scaled to hepatic intrinsic clearance (CLint,app (μL/min/million cells) using the following equation:

$$C {L}_{int , app} = ln 2 \cdot \frac{1}{{t}_{1 / 2} ( min )} \cdot \frac{mL incubation}{million cells in incubation }$$

Data obtained are shown in Table 5 below.

### Example EE. Neuropharmacokinetics Studies

The neuropharmacokinetics studies in mouse were conducted at Wuxi (Shanghai, China) with bioanalysis conducted at BioDuro (Beijing, China) with the exception of the Comparative-compound 3, which bioanalysis was conducted at Wuxi. All in vivo studies were conducted in accordance with regulations and established guidelines using protocols reviewed and approved by the Pfizer Worldwide Research and Development (WRD, or other) Institutional Animal Care and Use Committee.

### Mouse Subcutaneous Neuropharmacokinetics Studies

Male C57Bl6 mice were maintained on a 12-h light/dark cycle in a temperature- and humidity-controlled environment with free access to food and water. The test compounds (Examples 1 and 2 and Comparative Compounds 1-3) were administered as a subcutaneous dose of 1 mg/kg which was delivered in a dose volume of 10 mL/kg. The dose solution for all compounds was formulated in 5% DMSO/5% cremophor/90% saline. Blood and brain samples were collected at 0.5, 1, 2, 4, 8, 12, and 24 h post-dose (n=3 per time point). Blood samples were collected into tubes containing sodium fluoride and 4-(2-aminoethyl)benzenesulfonyl fluoride hydrochloride (AEBSF; final concentration 2 mg/mL) to inhibit hydrolase activity and placed on wet ice. Following centrifugation to afford plasma, the samples were transferred to polypropylene tubes and stored frozen at −20° C. to −80° C. until analysis. Immediately following blood collection the mice were euthanized by cervical dislocation and the brains were removed. The cerebellum was immediately frozen in liquid nitrogen and stored at −80° C. until analysis.

### Analysis of Compound in Plasma and Brain and Pharmacokinetic Parameter

Cerebellum samples were thawed, diluted 1:4 (w/v) with water, and homogenized using an H-speed dispersator. Standards and controls were prepared in a similar manner using a brain homogenate prepared from untreated animals. The plasma standard curves were generated with species specific plasma. All plasma and brain homogenate samples were quantified via LC/MS-MS using multiple reaction monitoring with non-validated methods.

### Nonspecific Binding Studies

Equilibrium dialysis using standard procedures [See Di et al., “Species independence in brain tissue binding using brain homogenates”; Drug Metab Dispos 39:1270-1277, 2011) were used for determination of the unbound fraction of the test compounds (Examples 1 and 2 and Comparative Compounds 1-3) in mouse plasma (f_{u,p}; see Di et al., “impact of recovery on fraction unbound using equilibrium dialysis”; J Pharm Sci 101:1327-1335, 2012) and unbound fraction in rat brain homogenate (f_{u,b}; see Di et al., “Species independence in brain tissue binding using brain homogenates”; Drug Metab Dispos 39:1270-1277, 2011). The determination of the unbound fraction in plasma also included the addition of AEBSF at a final concentration of 2 mg/mL to the incubation.

### Pharmacokinetic and Neuropharmacokinetic Calculations

The pharmacokinetic parameters were computed by non-compartmental analysis using Watson Bioanalytical LIMS version 7.5. The AUC values were calculated using the linear trapezoidal method for both brain and plasma. Measured total plasma (Cₚ) and brain (C_{b}; assuming brain tissue density of 1 g/mL) concentrations were converted to unbound (free) values using the matrix specific binding factor (f_{u, p} or f_{u,b}) to determine free plasma (C_{p,u}) and free brain (C_{b,u}) values. All neurocompartmental ratios (C_{b,u}/C_{p,u}) were calculated through use of AUC values with the exception of Comparative Compounds 1 and 2 which used the mean of individual animal values due to insufficient data to calculate an AUC value.

TABLE 5
Data of Examples BB, CC, DD, and EE.

| | HLM | HHEPs CLint, | | |
|---|---|---|---|---|
| | CLint, app | app | Mouse | Dog |
| Test Compound | (μL/min/mg) | (μL/min/million) | Cb,u/Cp,u^{a} | t_{1/2} (h) |
| 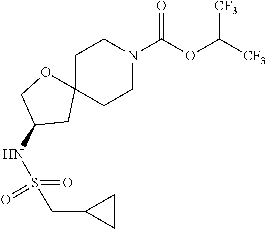 [SMILES: O=C(OC(C(F)(F)F)C(F)(F)F)N1CCC2(CC1)C[C@@H](NS(=O)(=O)CC1CC1)CO2] Example 1 | <8 | <4.26 | 1.0 | 16.9 |
| 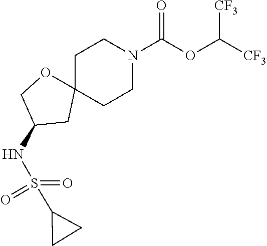 [SMILES: O=C(OC(C(F)(F)F)C(F)(F)F)N1CCC2(CC1)C[C@@H](NS(=O)(=O)C1CC1)CO2] Example 2 | <8 | 3.46 | 0.8 | 40.7 |
| 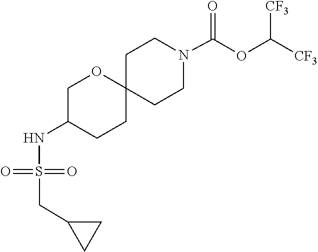 [SMILES: O=C(OC(C(F)(F)F)C(F)(F)F)N1CCC2(CCC(NS(=O)(=O)CC3CC3)CO2)CC1] Example 3 | <8 | <3.57 | NA | 26.3 |
| 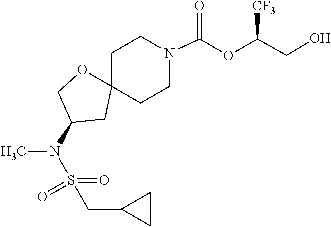 [SMILES: CN([C@H]1COC2(CCN(C(=O)O[C@H](CO)C(F)(F)F)CC2)C1)S(=O)(=O)CC1CC1] Comparative-Compound 1 | <12.5 | 5.5 | 0.18 | 0.50 |
| 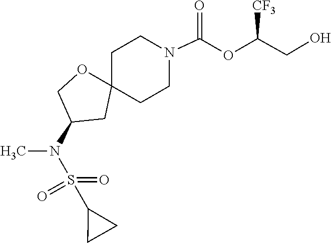 [SMILES: CN([C@H]1COC2(CCN(C(=O)O[C@H](CO)C(F)(F)F)CC2)C1)S(=O)(=O)C1CC1] Comparative-Compound 2 | <8 | <3.52 | 0.29 | 1.31 |
| 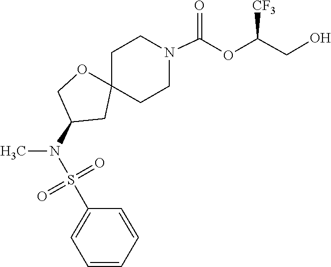 [SMILES: CN([C@H]1COC2(CCN(C(=O)O[C@H](CO)C(F)(F)F)CC2)C1)S(=O)(=O)c1ccccc1] Comparative-Compound 3 | 14.8 | 10.6 | 0.51 | 5.0 |
| 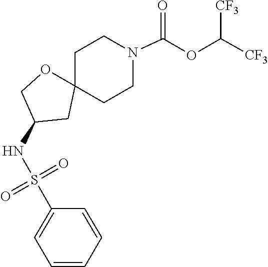 [SMILES: O=C(OC(C(F)(F)F)C(F)(F)F)N1CCC2(CC1)C[C@@H](NS(=O)(=O)c1ccccc1)CO2] Comparative-Compound 4 | 30.4 | 27.5 | N/A | N/A |

As shown in Table 5, the compounds of the present invention have increased brain penetration property and/or increased drug half-life property compared to the 1,1,1-trifluoro-3-hydroxypropan-2-yl compounds reported in U.S. Pat. No. 9,845,301.

Various modifications of the invention, in addition to those described herein, will be apparent to those skilled in the art from the foregoing description. Such modifications are also intended to fall within the scope of the appendant claims. Each reference (including all patents, patent applications, journal articles, books, and any other publications) cited in the present application is hereby incorporated by reference in its entirety.

1. A compound selected from the group consisting of:
1,1,1,3,3,3-hexafluoropropan-2-yl (3R)-3-{[(cyclopropylmethyl)sulfonyl]amino}-1-oxa-8-azaspiro[4.5]decane-8-carboxylate;
1,1,1,3,3,3-hexafluoropropan-2-yl (3R)-3-[(cyclopropylsulfonyl)amino]-1-oxa-8-azaspiro[4.5]decane-8-carboxylate;
1,1,1,3,3,3-hexafluoropropan-2-yl 3-{[(cyclopropylmethyl)sulfonyl]amino}-1-oxa-9-azaspiro[5.5]undecane-9-carboxylate;
1,1,1,3,3,3-hexafluoropropan-2-yl 3-{[(cyclopropylmethyl)sulfonyl]amino}-1-oxa-9-azaspiro[5.5]undecane-9-carboxylate, ENT-1; and
1,1,1,3,3,3-hexafluoropropan-2-yl 3-{[(cyclopropylmethyl)sulfonyl]amino}-1-oxa-9-azaspiro[5.5]undecane-9-carboxylate, ENT-2,
or a pharmaceutically acceptable salt thereof.

2. The compound of claim 1 that is 1,1,1,3,3,3-hexafluoropropan-2-yl (3R)-3-{[(cyclopropylmethyl)sulfonyl]amino}-oxa-8-azaspiro[4.5]decane-8-carboxylate, or a pharmaceutically acceptable salt thereof.

3. The compound of claim 1 that is 1,1,1,3,3,3-hexafluoropropan-2-yl (3R)-3-{[(cyclopropylmethyl)sulfonyl]amino}-oxa-8-azaspiro[4.5]decane-8-carboxylate.

4. A pharmaceutically acceptable salt of the compound of claim 1 that is 1,1,1,3,3,3-hexafluoropropan-2-yl (3R)-3-{[(cyclopropylmethyl)sulfonyl]amino}-oxa-8-azaspiro[4.5]decane-8-carboxylate.

5. The compound of claim 1 that is 1,1,1,3,3,3-hexafluoropropan-2-yl (3R)-3-[(cyclopropylsulfonyl)amino]-1-oxa-8-azaspiro[4.5]decane-8-carboxylate, or a pharmaceutically acceptable salt thereof.

6. The compound of claim 1 that is 1,1,1,3,3,3-hexafluoropropan-2-yl (3R)-3-[(cyclopropylsulfonyl)amino]-1-oxa-8-azaspiro[4.5]decane-8-carboxylate.

7. A pharmaceutically acceptable salt of the compound of claim 1 that is 1,1,1,3,3,3-hexafluoropropan-2-yl (3R)-3-[(cyclopropylsulfonyl)amino]-1-oxa-8-azaspiro[4.5]decane-8-carboxylate.

8. The compound of claim 1 that is 1,1,1,3,3,3-hexafluoropropan-2-yl 3-{[(cyclopropylmethyl)sulfonyl]amino}-1-oxa-9-azaspiro[5.5]undecane-9-carboxylate, or a pharmaceutically acceptable salt thereof.

9. The compound of claim 1 that is 1,1,1,3,3,3-hexafluoropropan-2-yl 3-{[(cyclopropylmethyl)sulfonyl]amino}-1-oxa-9-azaspiro[5.5]undecane-9-carboxylate.

10. A pharmaceutically acceptable salt of the compound of claim 1 that is 1,1,1,3,3,3-hexafluoropropan-2-yl 3-{[(cyclopropylmethyl)sulfonyl]amino}-1-oxa-9-azaspiro[5.5]undecane-9-carboxylate.

11. The compound of claim 1 that is 1,1,1,3,3,3-hexafluoropropan-2-yl 3-{[(cyclopropylmethyl)sulfonyl]amino}-1-oxa-9-azaspiro[5.5]undecane-9-carboxylate, ENT-1, or a pharmaceutically acceptable salt thereof.

12. The compound of claim 1 that is 1,1,1,3,3,3-hexafluoropropan-2-yl 3-{[(cyclopropylmethyl)sulfonyl]amino}-1-oxa-9-azaspiro[5.5]undecane-9-carboxylate, ENT-1.

13. A pharmaceutically acceptable salt of the compound of claim 1 that is 1,1,1,3,3,3-hexafluoropropan-2-yl 3-{[(cyclopropylmethyl)sulfonyl]amino}-1-oxa-9-azaspiro[5.5]undecane-9-carboxylate, ENT-1.

14. The compound of claim 1 that is 1,1,1,3,3,3-hexafluoropropan-2-yl 3-{[(cyclopropylmethyl)sulfonyl]amino}-1-oxa-9-azaspiro[5.5]undecane-9-carboxylate, ENT-2, or a pharmaceutically acceptable salt thereof.

15. The compound of claim 1 that is 1,1,1,3,3,3-hexafluoropropan-2-yl 3-{[(cyclopropylmethyl)sulfonyl]amino}-1-oxa-9-azaspiro[5.5]undecane-9-carboxylate, ENT-2.

16. A pharmaceutically acceptable salt of the compound of claim 1 that is 1,1,1,3,3,3-hexafluoropropan-2-yl 3-{[(cyclopropylmethyl)sulfonyl]amino}-1-oxa-9-azaspiro[5.5]undecane-9-carboxylate, ENT-2.

17-39. (canceled)

40. A pharmaceutical composition comprising (i) a compound according to claim 1, or pharmaceutically acceptable salt thereof; and (ii) a pharmaceutically acceptable carrier.

41-67. (canceled)

68. A method for treating a MAGL-mediated disease or disorder in a mammal, which method comprises administering to said mammal a therapeutically effective amount of a compound according to claim 1, or pharmaceutically acceptable salt thereof.

69. (canceled)

70. The method of claim 68, wherein the disorder is selected from the group consisting of a metabolic disorder (e.g., obesity); a kidney disease (e.g. acute inflammatory kidney injury and diabetic nephropathy); vomiting or emesis (e.g. chemotherapy induced vomiting); nausea (e.g. refractory nausea or chemotherapy induced nausea); an eating disorder (e.g., anorexia or bulimia); neuropathy (e.g., diabetic neuropathy, pellagric neuropathy, alcoholic neuropathy, Beriberi neuropathy); burning feet syndrome; a neurodegenerative disorder [multiple sclerosis (MS), Parkinson's disease (PD), Huntington's disease, dementia, Alzheimer's disease, amyotrophic lateral sclerosis (ALS), epilepsy, fronto-temporal lobe dementia, a sleep disorder, Creutzfeldt-Jakob disease (CJD), or prion disease]; a cardiovascular disease (e.g., hypertension, dyslipidemia, atherosclerosis, cardiac arrhythmias, or cardiac ischemia); osteoporosis; osteoarthritis; schizophrenia; depression; bipolar disease; tremor; dyskinesia; dystonia; spasticity; Tourette's syndrome; sleep apnea; hearing loss; an eye disease (e.g., glaucoma, ocular hypertension, macular degeneration, or a disease arising from elevated intraocular pressure); cachexia; insomnia; meningitis; sleeping sickness; progressive multifocal leukoencephalopathy; De Vivo disease; cerebral edema; cerebral palsy; withdrawal syndrome [alcohol withdrawal syndrome, antidepressant discontinuation syndrome, antipsychotic withdrawal syndrome, benzodiazepine withdrawal syndrome, cannabis withdrawal, neonatal withdrawal, nicotine withdrawal, or opioid withdrawal]; traumatic brain injury; non-traumatic brain injury; spinal cord injury; seizures; excitotoxin exposure; ischemia [stroke, hepatic ischemia or reperfusion, CNS ischemia or reperfusion]; liver fibrosis, iron overload, cirrhosis of the liver; a lung disorder [asthma, allergies, COPD, chronic bronchitis, emphysema, cystic fibrosis, pneumonia, tuberculosis, pulmonary edema, lung cancers, acute respiratory distress syndrome, intersitital lung disease (ILD), sarcoidosis, idiopathic pulmonary fibrosis, pulmonary embolism, pleural effusion, or mesothelioma]; a liver disorder [acute liver failure, Alagille syndrome, hepatitis, enlarged liver, Gilbert's syndrome, liver cysts, liver hemangioma, fatty liver disease, steatohepatitis [e.g. nonalcoholic Steatohepatitis (NASH)], primary sclerosing cholangitis, fascioliasis, primary bilary cirrhosis, Budd-Chiari syndrome, hemochromatosis, Wilson's disease, or transthyretin-related hereditary amyloidosis], stroke [e.g., ischemic stroke; hemorrhagic stroke]; subarachnoid hemorrhage; intracerebral hemorrhage; vasospasm; AIDS wasting syndrome; renal ischemia; a disorder associated with abnormal cell growth or proliferation [e.g., a benign tumor or cancer such as benign skin tumor, brain tumor, papilloma, prostate tumor, cerebral tumor (glioblastoma, medulloepithelioma, medulloblastoma, neuroblastoma, astrocytoma, astroblastoma, ependymoma, oligodendroglioma, plexus tumor, neuroepithelioma, epiphyseal tumor, ependymoblastoma, malignant meningioma, sarcomatosis, melanoma, schwannoma), melanoma, metastatic tumor, kidney cancer, bladder cancer, brain cancer, glioblastoma (GBM), gastrointestinal cancer, leukemia or blood cancer]; an autoimmune disease [e.g., psoriasis, lupus erythematosus, Sjogren's syndrome, ankylosing spondylitis, undifferentiated spondylitis, Behcet's disease, hemolytic anemia, graft rejection]; an inflammatory disorder [e.g., appendicitis, bursitis, colitis, cystitis, dermatitis, phlebitis, rhinitis, tendonitis, tonsillitis, vasculitis, acne vulgaris, chronic prostatitis, glomerulonephritis, hypersensitivities, IBS, pelvic inflammatory disease, sarcoidosis, HIV encephalitis, rabies, brain abscess, neuroinflammation, inflammation in the central nervous system (CNS)]; a disorder of the immune system (e.g., transplant rejection or celiac disease); post-traumatic stress disorder (PTSD); acute stress disorder; panic disorder; substance-induced anxiety; obsessive-compulsive disorder (OCD); agoraphobia; specific phobia; social phobia; anxiety disorder; attention deficit disorder (ADD); attention deficit hyperactivity disorder (ADHD); Asperger's syndrome; pain [e.g., acute pain; chronic pain; inflammatory pain; visceral pain; post-operative pain; migraine; lower back pain; joint pain; abdominal pain; chest pain; postmastectomy pain syndrome; menstrual pain; endometriosis pain; pain due to physical trauma; headache; sinus headache; tension headache arachnoiditis, herpes virus pain, diabetic pain; pain due to a disorder selected from: osteoarthritis, rheumatoid arthritis, osteoarthritis, spondylitis, gout, labor, musculoskeletal disease, skin disease, toothache, pyresis, burn, sunburn, snake bite, venomous snake bite, spider bite, insect sting, neurogenic bladder, interstitial cystitis, urinary tract infection (UTI), rhinitis, contact dermatitis/hypersensitivity, itch, eczema, pharyngitis, mucositis, enteritis, irritable bowel syndrome (IBS), cholecystitis, and pancreatitis; neuropathic pain (e.g., neuropathic low back pain, complex regional pain syndrome, post trigeminal neuralgia, causalgia, toxic neuropathy, reflex sympathetic dystrophy, diabetic neuropathy, chronic neuropathy from chemotherapeutic agent, or sciatica pain)]; a demyelinating disease [e.g., multiple sclerosis (MS), Devic's disease, CNS neuropathies, central pontine myelinolysis, syphilitic myelopathy, leukoencephalopathies, leukodystrophies, Guillain-Barre syndrome, chronic inflammatory demyelinating polyneuropathy, anti-myelin-associated glycoprotein (MAG) peripheral neuropathy, Charcot-Marie-Tooth disease, peripheral neuropathy, myelopathy, optic neuropathy, progressive inflammatory neuropathy, optic neuritis, transverse myelitis]; and cognitive impairment [e.g., cognitive impairment associated with Down's syndrome; cognitive impairment associated with Alzheimer's disease; cognitive impairment associated with PD; mild cognitive impairment (MCI), dementia, post-chemotherapy cognitive impairment (PCCI), postoperative cognitive dysfunction (POCD)].

71. (canceled)
